SEQUENCE LISTING (3 sequences)

SEQ ID NO: 1 - DNA, 7507 bp, Mus Musculus
  CDS (182)..(7507)
gctgttgctgaggctgagatttggccgccgcctcccccacccggcctgcgccctccgcggcccggcccgcgctcctgcgctcgctcctcgctggctcgcctgctcgcagccgccggcccgacccccgtccgggccgcgtcgcgccgcccgcgctcagggctgtttccgcgagcaggtgaagatggccgagaacttgctggacggaccgcccaaccccaaacgagccaaactcagctcgcccggcttctccgcgaatgacaacacagattttggatcattgtttgacttggaaaatgaccttcctgatgagctgatccccaatggagaattaagccttttaaacagtgggaaccttgttccagatgctgcgtccaaacataaacaactgtcagagcttcttagaggaggcagcggctctagcatcaacccagggataggcaatgtgagtgccagcagccctgtgcaacagggccttggtggccaggctcaggggcagccgaacagtacaaacatggccagcttaggtgccatgggcaagagccctctgaaccaaggagactcatcaacacccaacctgcccaaacaggcagccagcacctctgggcccactccccctgcctcccaagcactgaatccacaagcacaaaagcaagtagggctggtgaccagtagtcctgccacatcacagactggacctgggatctgcatgaatgctaacttcaaccagacccacccaggccttctcaatagtaactctggccatagcttaatgaatcaggctcaacaagggcaagctcaagtcatgaatggatctcttggggctgctggaagaggaaggggagctggaatgccctaccctgctccagccatgcagggggccacaagcagtgtgctggcggagaccttgacacaggtttccccacaaatggctggccatgctggactaaatacagcacaggcaggaggcatgaccaagatgggaatgactggtaccacaagtccatttggacaaccctttagtcaaactggagggcagcagatgggagccactggagtgaacccccagttagccagcaaacagagcatggtcaatagtttacctgcttttcctacagatatcaagaatacttcagtcaccactgtgccaaatatgtcccagttgcaaacatcagtgggaattgtacccacacaagcaattgcaacaggccccacagcagaccctgaaaaacgcaaactgatacagcagcagctggttctactgcttcatgcccacaaatgtcagagacgagagcaagcaaatggagaggttcgagcctgttctctcccacactgtcgaaccatgaaaaacgttttgaatcacatgacacattgtcaggctgggaaagcctgccaagttgcccattgtgcatcttcacgacaaatcatctctcattggaagaactgcacacgacatgactgtcctgtttgcctccctttgaaaaatgccagtgacaagcgaaaccaacaaaccatcctgggatctccagctagtggaattcaaaacacaattggttctgttggtgcagggcaacagaatgccacttccttaagtaacccaaatcccatagaccccagttccatgcagcgggcctatgctgctctaggactcccctacatgaaccagcctcagacgcagctgcagcctcaggttcctggccagcaaccagcacagcctccagcccaccagcagatgaggactctcaatgccctaggaaacaaccccatgagtatcccagcaggaggaataacaacagatcaacagccaccaaacttgatttcagaatcagctcttccaacttccttgggggctaccaatccactgatgaatgatggttcaaactctggtaacattggaagcctcagcacgatacctacagcagcgcctccttccagcactggtgttcgaaaaggctggcatgaacatgtgactcaggacctacggagtcatctagtccataaactcgttcaagccatcttcccaactccagaccctgcagctctgaaagatcgccgcatggagaacctggttgcctatgctaagaaagtggagggagacatgtatgagtctgctaatagcagggatgaatactatcatttattagcagagaaaatctataaaatacaaaaagaactagaagaaaagcggaggtcacgtttacataagcaaggcatcctgggtaaccagccagctttaccagcttctggggctcagccccctgtgattccaccagcccagtctgtaagacctccaaatgggcccctgcctttgccagtgaatcgcatgcaggtttctcaagggatgaattcatttaacccaatgtccctgggaaacgtccagttgccacaggcacccatgggacctcgtgcagcctcccctatgaaccactctgtgcagatgaacagcatggcctcagttccgggtatggccatttctccttcacggatgcctcagcctccaaatatgatgggcactcatgccaacaacattatggcccaggcacctactcagaaccagtttctgccacagaaccagtttccatcatccagtggggcaatgagtgtgaacagtgtgggcatggggcaaccagcagcccaggcaggtgtttcacagggtcaggtacctggagctgctctccctaaccctctgaacatgctggcaccccaggccagccagctgccttgcccaccagtgacacagtcaccattgcacccgactccacctcctgcttccacagctgctggcatgccctctctccaacatccaacggcaccaggaatgacccctcctcagccagcagctcccactcagccatctactcctgtgtcatctgggcagactcctaccccaactcctggctcagtgcccagcgctgcccaaacacagagtacccctacagtccaggcagcagcacaggctcaggtgactccacagcctcagaccccagtgcagccaccatctgtggctactcctcagtcatcacagcagcaaccaacgcctgtgcatactcagcctcctggcacaccgctttctcaggcagcagccagcattgataatagagtccctactccctcctctgtgaccagtgctgaaaccagttcccagcagccaggacccgatgtgcccatgctggaaatgaagacagaggtgcagacagatgatgctgagcctgaacctactgaatccaagggggaacctcggtctgagatgatggaagaggatttacaaggttcttcccaagtaaaagaagagacagatacgacagagcagaagtcagagccaatggaagtagaagaaaagaaacctgaagtaaaagtggaagctaaagaggaagaagagaacagttcgaacgacacagcctcacaatcaacatctccttcccagccacgcaaaaaaatctttaaacccgaggagctacgccaggcacttatgccaactctagaagcactctatcgacaggacccagagtctttgccttttcgtcagcctgtagatcctcagctcctaggaatcccagattattttgatatagtgaagaatcctatggacctttctaccatcaaacgaaagctggacacagggcaatatcaagaaccctggcagtatgtggatgatgtctggcttatgttcaacaatgcgtggctatataatcgtaaaacgtcccgtgtatataaattttgcagtaaacttgcagaggtctttgaacaagaaattgaccctgtcatgcagtctcttggatattgctgtggacgaaagtatgagttctccccacagactttgtgctgttacggaaagcagctgtgtacaattcctcgtgatgcagcctactacagctatcagaataggtatcatttctgtgagaagtgtttcacagagatccagggcgagaatgtgaccctgggtgacgacccttcccaacctcagacgacaatttccaaggatcaatttgaaaagaagaaaaatgataccttagatcctgaaccttttgttgactgcaaagagtgtggccggaagatgcatcagatttgtgttctacactatgacatcatttggccttcaggttttgtgtgtgacaactgtttgaagaaaactggcagacctcggaaagaaaacaaattcagtgctaagaggctgcagaccacacgattgggaaaccacttagaagacagagtgaataagtttttgcggcgccagaatcaccctgaagctggggaggtttttgtcagagtggtggccagctcagacaagactgtggaggtcaagccgggaatgaagtcaaggtttgtggattctggagagatgtcggaatctttcccatatcgtaccaaagcactctttgcttttgaggagatcgatggagtcgatgtgtgcttttttgggatgcatgtgcaagaatacggctctgattgccccccaccaaatacaaggcgtgtatacatatcttatctggacagtattcatttcttccggccccgctgcctccggacagctgtttaccatgagatcctcatcggatatctcgagtatgtgaagaaattggggtatgtgacaggacatatttgggcctgtcccccaagtgaaggagatgactatatctttcattgccacccccctgaccagaaaatccccaaaccaaaacgactacaggagtggtacaagaagatgctggacaaggcgtttgcagagaggatcattaacgactataaggacatcttcaaacaagcgaacgaagacaggctcacgagtgccaaggagttgccctattttgaaggagatttctggcctaatgtgttggaagaaagcattaaggaactagaacaagaagaagaagaaaggaaaaaagaagagagtactgcagcgagtgagactcctgagggcagtcagggtgacagcaaaaatgcgaagaaaaagaacaacaagaagaccaacaaaaacaaaagcagcattagccgcgccaacaagaagaagcccagcatgcccaatgtttccaacgacctgtcgcagaagctgtatgccaccatggagaagcacaaggaggtattctttgtgattcatctgcatgctgggcctgttatcagcactcagccccccatcgtggaccctgatcctctgcttagctgtgacctcatggatgggcgagatgccttcctcaccctggccagagacaagcactgggaattctcttccttacgccgctccaaatggtccactctgtgcatgctggtggagctgcacacacagggccaggaccgctttgtttatacctgcaatgagtgcaaacaccatgtggaaacacgctggcactgcactgtgtgtgaggactatgacctttgtatcaattgctacaacacaaagagccacacccataagatggtgaagtgggggctaggcctagatgatgagggcagcagtcagggtgagccacagtccaagagcccccaggaatcccggcgtctcagcatccagcgctgcatccagtccctggtgcatgcctgccagtgtcgcaatgccaactgctcactgccgtcttgccagaagatgaagcgagtcgtgcagcacaccaagggctgcaagcgcaagactaatggaggatgcccagtgtgcaagcagctcattgctctttgctgctaccacgccaaacactgccaagaaaataaatgccctgtgcccttctgcctcaacatcaaacataagctccgccagcagcagatccagcatcgcctgcagcaggctcagctcatgcgccggcgaatggcaaccatgaacacccgcaatgtgcctcagcagagtttgccttctcctacctcagcaccacccgggactcctacacagcagcccagcacaccccaaacaccacagcccccagcccagcctcagccttcacctgttaacatgtcaccagctggcttccctaatgtagcccggactcagcccccaacaatagtgtctgctgggaagcctaccaaccaggtgccagctcccccaccccctgcccagcccccacctgcagcagtagaagcagcccggcaaattgaacgtgaggcccagcagcagcagcacctataccgagcaaacatcaacaatggcatgcccccaggacgtgcaggtatggggaccccaggaagccaaatgactcctgtgggcctgaatgtgccccgtcccaaccaagtcagtgggcctgtcatgtctagtatgccacctgggcagtggcagcaggcacccatccctcagcagcagccgatgccaggcatgcccaggcctgtaatgtccatgcaggcccaggcagcagtggctgggccacggatgcccaatgtgcagccaccaaggagcatctcgccaagtgccctgcaagacctgctacggaccctaaagtcacccagctctcctcagcagcagcagcaggtgctgaacatccttaaatcaaacccacagctaatggcagctttcatcaaacagcgcacagccaagtatgtggccaatcagcctggcatgcagccccagcccggacttcaatcccagcctggtatgcagccccagcctggcatgcaccagcagcctagtttgcaaaacctgaacgcaatgcaagctggtgtgccacggcctggtgtgcctccaccacaaccagcaatgggaggcctgaatccccagggacaagctctgaacatcatgaacccaggacacaaccccaacatgacaaacatgaatccacagtaccgagaaatggtgaggagacagctgctacagcaccagcagcagcagcagcaacagcagcagcagcagcagcaacaacaaaatagtgccagcttggccgggggcatggcgggacacagccagttccagcagccacaaggacctggaggttatgccccagccatgcagcagcaacgcatgcaacagcacctccccatccagggcagctccatgggccagatggctgctccaatgggacaacttggccagatggggcagcctgggctaggggcagacagcacccctaatatccagcaggccctgcagcaacggattctgcagcagcagcagatgaagcaacaaattgggtcaccaggccagccgaaccccatgagcccccagcagcacatgctctcaggacagccacaggcctcacatctccctggccagcagatcgccacatcccttagtaaccaggtgcgatctccagcccctgtgcagtctccacggccccaatcccaacctccacattccagcccgtcaccacggatacaaccccagccttcaccacaccatgtttcaccccagactggttcccctcaccctggactcgcagtcaccatggccagctccatggatcagggacacctggggaaccctgaacagagtgcaatgctcccccagctgaatacccccaacaggagcgcactgtccagtgaactgtccctggttggtgataccacgggagacacactagaaaagtttgtggagggtttgtag

SEQ ID NO: 2 - protein, 2441 aa, Mus Musculus
MAENLLDGPPNPKRAKLSSPGFSANDNTDFGSLFDLENDLPDELIPNGELSLLNSGNLVPDAASKHKQLSELLRGGSGSSINPGIGNVSASSPVQQGLGGQAQGQPNSTNMASLGAMGKSPLNQGDSSTPNLPKQAASTSGPTPPASQALNPQAQKQVGLVTSSPATSQTGPGICMNANFNQTHPGLLNSNSGHSLMNQAQQGQAQVMNGSLGAAGRGRGAGMPYPAPAMQGATSSVLAETLTQVSPQMAGHAGLNTAQAGGMTKMGMTGTTSPFGQPFSQTGGQQMGATGVNPQLASKQSMVNSLPAFPTDIKNTSVTTVPNMSQLQTSVGIVPTQAIATGPTADPEKRKLIQQQLVLLLHAHKCQRREQANGEVRACSLPHCRTMKNVLNHMTHCQAGKACQVAHCASSRQIISHWKNCTRHDCPVCLPLKNASDKRNQQTILGSPASGIQNTIGSVGAGQQNATSLSNPNPIDPSSMQRAYAALGLPYMNQPQTQLQPQVPGQQPAQPPAHQQMRTLNALGNNPMSIPAGGITTDQQPPNLISESALPTSLGATNPLMNDGSNSGNIGSLSTIPTAAPPSSTGVRKGWHEHVTQDLRSHLVHKLVQAIFPTPDPAALKDRRMENLVAYAKKVEGDMYESANSRDEYYHLLAEKIYKIQKELEEKRRSRLHKQGILGNQPALPASGAQPPVIPPAQSVRPPNGPLPLPVNRMQVSQGMNSFNPMSLGNVQLPQAPMGPRAASPMNHSVQMNSMASVPGMAISPSRMPQPPNMMGTHANNIMAQAPTQNQFLPQNQFPSSSGAMSVNSVGMGQPAAQAGVSQGQVPGAALPNPLNMLAPQASQLPCPPVTQSPLHPTPPPASTAAGMPSLQHPTAPGMTPPQPAAPTQPSTPVSSGQTPTPTPGSVPSAAQTQSTPTVQAAAQAQVTPQPQTPVQPPSVATPQSSQQQPTPVHTQPPGTPLSQAAASIDNRVPTPSSVTSAETSSQQPGPDVPMLEMKTEVQTDDAEPEPTESKGEPRSEMMEEDLQGSSQVKEETDTTEQKSEPMEVEEKKPEVKVEAKEEEENSSNDTASQSTSPSQPRKKIFKPEELRQALMPTLEALYRQDPESLPFRQPVDPQLLGIPDYFDIVKNPMDLSTIKRKLDTGQYQEPWQYVDDVWLMFNNAWLYNRKTSRVYKFCSKLAEVFEQEIDPVMQSLGYCCGRKYEFSPQTLCCYGKQLCTIPRDAAYYSYQNRYHFCEKCFTEIQGENVTLGDDPSQPQTTISKDQFEKKKNDTLDPEPFVDCKECGRKMHQICVLHYDIIWPSGFVCDNCLKKTGRPRKENKFSAKRLQTTRLGNHLEDRVNKFLRRQNHPEAGEVFVRVVASSDKTVEVKPGMKSRFVDSGEMSESFPYRTKALFAFEEIDGVDVCFFGMHVQEYGSDCPPPNTRRVYISYLDSIHFFRPRCLRTAVYHEILIGYLEYVKKLGYVTGHIWACPPSEGDDYIFHCHPPDQKIPKPKRLQEWYKKMLDKAFAERIINDYKDIFKQANEDRLTSAKELPYFEGDFWPNVLEESIKELEQEEEERKKEESTAASETPEGSQGDSKNAKKKNNKKTNKNKSSISRANKKKPSMPNVSNDLSQKLYATMEKHKEVFFVIHLHAGPVISTQPPIVDPDPLLSCDLMDGRDAFLTLARDKHWEFSSLRRSKWSTLCMLVELHTQGQDRFVYTCNECKHHVETRWHCTVCEDYDLCINCYNTKSHTHKMVKWGLGLDDEGSSQGEPQSKSPQESRRLSIQRCIQSLVHACQCRNANCSLPSCQKMKRVVQHTKGCKRKTNGGCPVCKQLIALCCYHAKHCQENKCPVPFCLNIKHKLRQQQIQHRLQQAQLMRRRMATMNTRNVPQQSLPSPTSAPPGTPTQQPSTPQTPQPPAQPQPSPVNMSPAGFPNVARTQPPTIVSAGKPTNQVPAPPPPAQPPPAAVEAARQIEREAQQQQHLYRANINNGMPPGRAGMGTPGSQMTPVGLNVPRPNQVSGPVMSSMPPGQWQQAPIPQQQPMPGMPRPVMSMQAQAAVAGPRMPNVQPPRSISPSALQDLLRTLKSPSSPQQQQQVLNILKSNPQLMAAFIKQRTAKYVANQPGMQPQPGLQSQPGMQPQPGMHQQPSLQNLNAMQAGVPRPGVPPPQPAMGGLNPQGQALNIMNPGHNPNMTNMNPQYREMVRRQLLQHQQQQQQQQQQQQQQQNSASLAGGMAGHSQFQQPQGPGGYAPAMQQQRMQQHLPIQGSSMGQMAAPMGQLGQMGQPGLGADSTPNIQQALQQRILQQQQMKQQIGSPGQPNPMSPQQHMLSGQPQASHLPGQQIATSLSNQVRSPAPVQSPRPQSQPPHSSPSPRIQPQPSPHHVSPQTGSPHPGLAVTMASSMDQGHLGNPEQSAMLPQLNTPNRSALSSELSLVGDTTGDTLEKFVEGL

SEQ ID NO: 3 - protein, 2442 aa, Homo sapiens
MAENLLDGPPNPKRAKLSSPGFSANDSTDFGSLFDLENDLPDELIPNGGELGLLNSGNLVPDAASKHKQLSELLRGGSGSSINPGIGNVSASSPVQQGLGGQAQGQPNSANMASLSAMGKSPLSQGDSSAPSLPKQAASTSGPTPAASQALNPQAQKQVGLATSSPATSQTGPGICMNANFNQTHPGLLNSNSGHSLINQASQGQAQVMNGSLGAAGRGRGAGMPYPTPAMQGASSSVLAETLTQVSPQMTGHAGLNTAQAGGMAKMGITGNTSPFGQPFSQAGGQPMGATGVNPQLASKQSMVNSLPTFPTDIKNTSVTNVPNMSQMQTSVGIVPTQAIATGPTADPEKRKLIQQQLVLLLHAHKCQRREQANGEVRACSLPHCRTMKNVLNHMTHCQAGKACQVAHCASSRQIISHWKNCTRHDCPVCLPLKNASDKRNQQTILGSPASGIQNTIGSVGTGQQNATSLSNPNPIDPSSMQRAYAALGLPYMNQPQTQLQPQVPGQQPAQPQTHQQMRTLNPLGNNPMNIPAGGITTDQQPPNLISESALPTSLGATNPLMNDGSNSGNIGTLSTIPTAAPPSSTGVRKGWHEHVTQDLRSHLVHKLVQAIFPTPDPAALKDRRMENLVAYAKKVEGDMYESANSRDEYYHLLAEKIYKIQKELEEKRRSRLHKQGILGNQPALPAPGAQPPVIPQAQPVRPPNGPLSLPVNRMQVSQGMNSFNPMSLGNVQLPQAPMGPRAASPMNHSVQMNSMGSVPGMAISPSRMPQPPNMMGAHTNNMMAQAPAQSQFLPQNQFPSSSGAMSVGMGQPPAQTGVSQGQVPGAALPNPLNMLGPQASQLPCPPVTQSPLHPTPPPASTAAGMPSLQHTTPPGMTPPQPAAPTQPSTPVSSSGQTPTPTPGSVPSATQTQSTPTVQAAAQAQVTPQPQTPVQPPSVATPQSSQQQPTPVHAQPPGTPLSQAAASIDNRVPTPSSVASAETNSQQPGPDVPVLEMKTETQAEDTEPDPGESKGEPRSEMMEEDLQGASQVKEETDIAEQKSEPMEVDEKKPEVKVEVKEEEESSSNGTASQSTSPSQPRKKIFKPEELRQALMPTLEALYRQDPESLPFRQPVDPQLLGIPDYFDIVKNPMDLSTIKRKLDTGQYQEPWQYVDDVWLMFNNAWLYNRKTSRVYKFCSKLAEVFEQEIDPVMQSLGYCCGRKYEFSPQTLCCYGKQLCTIPRDAAYYSYQNRYHFCEKCFTEIQGENVTLGDDPSQPQTTISKDQFEKKKNDTLDPEPFVDCKECGRKMHQICVLHYDIIWPSGFVCDNCLKKTGRPRKENKFSAKRLQTTRLGNHLEDRVNKFLRRQNHPEAGEVFVRVVASSDKTVEVKPGMKSRFVDSGEMSESFPYRTKALFAFEEIDGVDVCFFGMHVQEYGSDCPPPNTRRVYISYLDSIHFFRPRCLRTAVYHEILIGYLEYVKKLGYVTGHIWACPPSEGDDYIFHCHPPDQKIPKPKRLQEWYKKMLDKAFAERIIHDYKDIFKQATEDRLTSAKELPYFEGDFWPNVLEESIKELEQEEEERKKEESTAASETTEGSQGDSKNAKKKNNKKTNKNKSSISRANKKKPSMPNVSNDLSQKLYATMEKHKEVFFVIHLHAGPVINTLPPIVDPDPLLSCDLMDGRDAFLTLARDKHWEFSSLRRSKWSTLCMLVELHTQGQDRFVYTCNECKHHVETRWHCTVCEDYDLCINCYNTKSHAHKMVKWGLGLDDEGSSQGEPQSKSPQESRRLSIQRCIQSLVHACQCRNANCSLPSCQKMKRVVQHTKGCKRKTNGGCPVCKQLIALCCYHAKHCQENKCPVPFCLNIKHKLRQQQIQHRLQQAQLMRRRMATMNTRNVPQQSLPSPTSAPPGTPTQQPSTPQTPQPPAQPQPSPVSMSPAGFPSVARTQPPTTVSTGKPTSQVPAPPPPAQPPPAAVEAARQIEREAQQQQHLYRVNINNSMPPGRTGMGTPGSQMAPVSLNVPRPNQVSGPVMPSMPPGQWQQAPLPQQQPMPGLPRPVISMQAQAAVAGPRMPSVQPPRSISPSALQDLLRTLKSPSSPQQQQQVLNILKSNPQLMAAFIKQRTAKYVANQPGMQPQPGLQSQPGMQPQPGMHQQPSLQNLNAMQAGVPRPGVPPQQQAMGGLNPQGQALNIMNPGHNPNMASMNPQYREMLRRQLLQQQQQQQQQQQQQQQQQQGSAGMAGGMAGHGQFQQPQGPGGYPPAMQQQQRMQQHLPLQGSSMGQMAAQMGQLGQMGQPGLGADSTPNIQQALQQRILQQQQMKQQIGSPGQPNPMSPQQHMLSGQPQASHLPGQQIATSLSNQVRSPAPVQSPRPQSQPPHSSPSPRIQPQPSPHHVSPQTGSPHPGLAVTMASSIDQGHLGNPEQSAMLPQLNTPSRSALSSELSLVGDTTGDTLEKFVEGL

(19) United States
(12) Patent Application Publication
(10) Pub. No.: US 2016/0008489 A1
(43) Pub. Date: Jan. 14, 2016

(54) METHODS FOR MODULATING NUCLEAR ACETYLTRANSFERASE ACTIVITY IN LIVING BRAIN, MEMORY ACCURACY AND FEAR GENERALIZATION

(71) Applicant: THE REGENTS OF THE UNIVERSITY OF CALIFORNIA, Oakland, CA (US)

(72) Inventor: Edward KORZUS, Riverside, CA (US)

(21) Appl. No.: 14/798,704

(22) Filed: Jul. 14, 2015

Related U.S. Application Data
(60) Provisional application No. 62/024,181, filed on Jul. 14, 2014.

(51) Int. Cl. A61K 49/00

(52) U.S. Cl. CPC A61K 49/0008

(57) ABSTRACT

The disclosure provides methods for screening for a modifier or a modulator of a brain function or a cognitive function. The disclosure also provides methods for modifying or modulating a brain function or a cognitive function in an individual.

### CROSS REFERENCE TO RELATED APPLICATIONS

The application claims priority under 35 U.S.C. §119 to U.S. Provisional Application Ser. No. 62/024,181, filed Jul. 14, 2014, the disclosure of which is incorporated herein by reference.

### STATEMENT AS TO FEDERALLY SPONSORED RESEARCH

This invention was made with government support under Grant No. MH086078 awarded by the NIMH, National Institutes of Health. The government has certain rights in the invention.

### TECHNICAL FIELD

This disclosure generally relates to drug discovery and medical diagnostics. In alternative embodiments, the invention provides methods for screening for a modifier or a modulator of a brain function or a cognitive function.

### BACKGROUND

The ability to discriminate between similar, yet different, stimuli is important for cognitive functioning and is referred to as memory specificity or memory accuracy. Failure to discriminate between aversive and non-aversive stimuli during recall may indicate decreased memory resolution (i.e., reduced access to memory details) or generalized fear or both, and may lead to inappropriate stimulus generalization. Generalization is not always inappropriate and this type of reduced fear memory accuracy is observed when one responds the same to two stimuli that are not identical. After initial generalization, fear memory accuracy can be increased through additional experiences with reinforced aversive stimulus and non-reinforced non-aversive stimulus. Conversely, over-generalized fear is a typical symptom of anxiety disorders including phobias and posttraumatic stress disorder (PTSD), which are triggered by cues resembling traumatic experience in a secure environment. Studies of neural substrates and mechanisms underlying memory resolution are focused on the hippocampal circuit. Recent studies also implicate prefrontal circuitry in the contextual fear memory specificity and generalization or discrimination of more discrete multiple odor stimuli.

Regulatory mechanisms direct cAMP response element binding protein (CREB)-dependent transcription subsequent to learning-induced molecular changes in which neurons play a pivotal role in the conversion of short-term to long-term memory across species. Phosphorylation of CREB at serine 133 is required for the recruitment of the chromatin remodeling factor with intrinsic acetyltransferase activity CREB binding protein (CBP), both events are important for CREB-dependent transcription. CBP integrates multiple signaling pathways via direct interactions with independently regulated multiple transcriptional factors and components of transcriptional machinery. In addition, CBP comprises enzymatic activity referred to as HAT (histone acetyltransferase), which enables acetylation of conserved lysine amino acids on proteins by catalyzing a transfer of an acetyl group of acetyl CoA to form ε-N-acetyl-lysine. Initially histones were considered as primary natural substrates for CBP enzymatic activity. However histones are not the only targets for CBP's HAT activity and a number of non-histone potential targets for CBP's HAT activity have been found, including proteins regulating chromatin remodeling and gene expression such as p53 and CREB. Impact of histone and non-histone protein acetylation by CBP is not fully understood.

A role for CBP in higher cognitive function is suggested by the finding that Rubinsten-Taybi syndrome (RTS), a disorder in humans characterized by growth and psychomotor delay, abnormal gross anatomy and severe mental retardation, is caused by heterozygous mutations at the CBP locus. However, because of the complexity of developmental abnormalities and possible genetic compensation associated with this congenital disorder, it is difficult to establish a direct role for CBP in cognitive function in the adult brain.

Although there has been extensive research into the function of the PFC during information acquisition and retrieval, a fundamental question that has escaped resolution is whether CBP-dependent signaling within the prefrontal cortex supports mechanisms in which fear memories are encoded and retrieved without confusion.

### SUMMARY

In alternative embodiments, the invention provides methods for screening for a modifier or a modulator of a brain function or a cognitive function. In alternative embodiments, the invention provides methods for modifying or modulating a brain function or a cognitive function in an individual.

In alternative embodiments, the invention provides methods for screening for a modifier or a modulator of a brain function or a cognitive function, comprising (a) providing a non-human animal having a dysfunctional, non-functional, or partially, substantially or completely disabled cAMP response element binding protein (CREB) binding protein (CBP) or equivalent cellular transcriptional coactivators, wherein optionally the brain function or cognitive function comprises information acquisition capability, short-term or long-term memory, a memory consolidation, a memory accuracy, a memory generalization, a fear generalization, a contextual discrimination, an auditory memory or an auditory discrimination, wherein optionally the non-human animal is a transgenic non-human animal, or a chemically or genetically modified non-human animal, and optionally the non-human animal is a mouse or a rat, wherein optionally the CBP protein function is partially, substantially or completely disabled by at least one mutation in the CBP gene, and optionally the at least one mutation in the CBP gene comprises at least one mutation in the histone acetyltransferase (HAT) domain of the CBP protein-encoding gene, and optionally the at least one mutation in the CBP gene comprises at least one mutation in the lysine acetyltransferase (KAT) domain of the CBP protein-encoding gene, and optionally the mutation results in a CBP that has no intrinsic acetyltransferase activity due to its inability to interact with a donor of acetyl group, acetyl-CoA but retains all protein-protein interaction domains, and optionally the at least one mutation in the acetyltransferase domain comprises a substitution of residue 1540 or 1541 of SEQ ID NO:2, or equivalent, wherein optionally the substitution of residue 1540 or 1541 of SEQ ID NO:2, comprises a Tyr¹⁵⁴⁰/Phe¹⁵⁴¹ to Ala¹⁵⁴⁰/Ala¹⁵⁴¹ in the acetyl CoA binding domain; (b) providing a test compound, wherein optionally the test compound is a small molecule, a lipid, a nucleic acid, a polysaccharide, peptide or a protein, and optionally the nucleic acid comprises an antisense nucleic acid, or an siRNA or an miRNA; and (c) administering the test compound to the non-human animal, and testing or determining if the animal has any change in a brain function or a cognitive function, wherein optionally the brain function or cognitive function comprises information acquisition capability, short-term or long-term memory, a memory consolidation, a memory accuracy, a memory generalization, a fear generalization, a contextual discrimination, an auditory memory or an auditory discrimination, wherein optionally determining if the animal has any change in a brain function or a cognitive function is accomplished by using a behavioral test or an empirical measurement, wherein optionally the behavioral test comprises a generalization task or a context fear discrimination task, or a Pavlovian auditory or a contextual fear conditioning, wherein optionally the empirical measurement comprises use of: a Magnetic resonance imaging (MRI), a nuclear magnetic resonance imaging (NMRI), a magnetic resonance tomography (MRT), a Functional magnetic resonance imaging or functional MRI (fMRI), a Positron emission tomography (PET), a Positron emission tomography-computed tomography (PET-CT or PET/CT), a Electroencephalography (EEG), an Electronystagmography (ENG), or a Magnetoencephalography (MEG), to determine any change in a brain function or a cognitive function, wherein a finding that the test compound modifies or modulates the brain function or the cognitive function identifies the test compound as a modifier or a modulator of a brain function or a cognitive function.

In alternative embodiments, the invention provides methods for modifying or modulating a brain function or a cognitive function in an individual, comprising generating: (a) a dysfunctional, non-functional, or partially, substantially or completely disabled cyclic AMP-response element binding (CBP) protein or equivalent cellular transcriptional coactivator, or inducing non-expression or dysfunctional expression of, or a dysfunction or non-function in, a CBP protein or equivalent cellular transcriptional coactivator, or (b) a dysfunctional, non-functional, or partially, substantially or completely disabled nuclear acetyltransferase or histone acetyltransferase, or inducing non-expression or dysfunctional expression of, or a dysfunction or non-function in, a nuclear acetyltransferase or histone acetyltransferase, by administering a compound or composition or by genetic manipulation of the individual, wherein optionally compound or composition comprises a small molecule, a lipid, a nucleic acid, a polysaccharide, peptide or a protein, and optionally the nucleic acid comprises an antisense nucleic acid, or an siRNA or an miRNA, and optionally the peptide or a protein comprise an antibody or an antigen binding fragment thereof, wherein optionally the brain function comprises a cognitive function, wherein optionally the cognitive function comprises information acquisition capability, short-term or long-term memory, a memory consolidation, a memory accuracy, a memory generalization, a fear generalization, a contextual discrimination, an auditory memory or an auditory discrimination, wherein optionally the individual is a human or a non-human animal, or the individual is a chemically modified human or a non-human animal, and optionally the non-human animal is a transgenic non-human animal, or a chemically or genetically modified non-human animal, and optionally the non-human animal is a mouse or a rat, wherein optionally the CBP protein function is partially, substantially or completely disabled by at least one mutation in the CBP gene, and optionally the at least one mutation in the CBP gene comprises at least one mutation in the histone acetyltransferase (HAT) domain of the CBP protein-encoding gene, and optionally the mutation results in a CBP that has no intrinsic acetyltransferase activity due to its inability to interact with a donor of acetyl group, acetyl-CoA but retains all protein-protein interaction domains, and optionally the at least one mutation in the histone acetyltransferase (HAT) domain comprises a substitution of residue 1540 or 1541 of SEQ ID NO:2, or equivalent, wherein optionally the substitution of residue 1540 or 1541 of SEQ ID NO:2, comprises a Tyr¹⁵⁴⁰/Phe¹⁵⁴¹ to Ala¹⁵⁴⁰/Ala¹⁵⁴¹ in the acetyl CoA binding domain.

The disclosure provides an assay for screening modulators of cognitive function comprising (a) administering the modulator to a transgenic animal subject, wherein the transgenic animal subject has at least one mutation in the histone acetyltransferase (HAT) domain of the cyclic amp-response element binding protein (CBP) enzyme; and (b) monitoring cognitive function of the animal subject after administration. In one embodiment, the transgenic animal subject has at least one substitution mutation in the histone acetyltransferase domain. In another embodiment, the transgenic animal subject has a substitute mutation at residues 1540 or 1541. In yet another embodiment, the cognitive function is memory consolidation. In another embodiment, the cognitive function is memory accuracy. In still another embodiment, the cognitive function is memory generalization. In yet another embodiment, the cognitive function is fear generalization. In one embodiment, the cognitive function is contextual discrimination. In another embodiment, the cognitive function is auditory discrimination.

The disclosure also provides a method of altering cognitive function comprising selectively modulating nuclear acetyltransferase activity of a nuclear protein. In one embodiment, the modulation is direct. In another embodiment, the modulation is indirect. In yet another embodiment, the modulation is antibody mediated. In still another embodiment, the modulation is gene mediated. In one embodiment, the modulation is small molecule mediated.

The disclosure also provides assays and animal models for testing testing cognitive function in pharmacological and genetic models. In one embodiment, the cognitive function is memory consolidation. In another embodiment, the cognitive function is memory accuracy. In yet another embodiment, the cognitive function is memory generalization. In still another embodiment, the cognitive function is fear generalization. In another embodiment, the cognitive function is contextual discrimination. In still yet another embodiment, the cognitive function is auditory discrimination.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

The drawings set forth herein are illustrative of embodiments of the invention and are not meant to limit the scope of the invention as encompassed by the claims.

FIG. 1A-J shows that contextual fear memory specificity was deficient in CBPΔHAT^{PFC} mice. (A) Shows viral-mediated delivery to the medial prefrontal cortex (mPFC). Long-term expression HSV-1 viruses carrying CBPΔHAT (HSV/CBPΔHAT-IRES2-EGFP) or eGFP as the control (HSV/EGFP) were injected into the mouse mPFC. To determine the pattern of GFP-tagged virus expression, the imaged tissue was compared to the Paxinos and Franklin (2001) mouse atlas and areas of maximal GFP expression were labeled as injection sites. A representative image of mPFC viral infection showed the precision of the viral-targeting procedures. The pattern of EGFP expression was similar 4 d or 20 d after HSV virus injection into the mPFC. (shown are GFP; and white, NeuN neuronal marker). (B) CBPΔHAT blocks acetylation of histone H3 and H4 in the mPFC. To determine the effects of viral infection with CBPΔHAT on neural signaling, the levels of acetylated histones H3 and H4 were assessed in the brains of infected animals and compared to controls in a standard IHC analysis 25 min after auditory fear conditioning. Cells expressing viral CBPΔHAT showed significantly lower levels of acetylated histone H3 and H4 when compared to control animals expressing GFP only. Representative images show GFP and acetylated histone H3 (Ac-H3, left panel; t-test, t₍₁₀₎=2.38, P=0.0382, effect size r=0.6013) or acetylated histone H4 (Ac-H4, right panel; t-test, t₍₁₀₎=2.9718, P=0.0140, effect size r=0.6848) CBPΔHAT^{PFC} mice. Three animals were used per group. GFP, green; Ac-H3, white; Ac-H4, white; bar, 10 mm. (C) Pavlovian contextual fear conditioning was normal in CBPΔHAT^{PFC} mice. CBPΔHAT^{PFC} and control (Ctrl) mice showed normal acquisition and retention of contextual fear conditioning. Contextual fear was tested in context A at 24 h after a single context A-foot shock pairing. (D) Experimental design for the context discrimination test. Context A and B were similar but not identical. The protocol included 14 d of training. The mice were placed in context A (CS+) for 180 sec followed by a foot shock (arrow), and context B (CS−) lacked any reinforcement. (E) Generalization test. Freezing behavior to context A and a similar but not identical context B after conditioning to context A-foot shock pairing was not different, in both groups. Freezing in both tested groups was comparable in response to both contexts, indicating that context A was sufficiently similar to context B that generalization was occurring early in training. (F) After the initial generalization of fear conditioned responses, control mice exhibited robust fear memory specificity. (E) CBPΔHAT^{PFC} mice exhibited a deficit in context discrimination. (H) The context discrimination ratios (DI) were calculated using the freezing responses to CS+ and CS− according to the formula DI=([Context A 2 Context B]/[Context A+Context B]). Analyses revealed differences in the performance during Trial Block 6 between CBPΔHAT^{PFC} and control mice, but not during Trial Blocks 1-5. CBPΔHAT^{PFC} mice, n=11. Control, n=9. (I) Change in freezing across training (freezing A), calculated as the (freezing on Trial Block 6—freezing on Trial Block 1). There was no difference in responses to conditioned stimuli CS+ between CBPΔHAT^{PFC} and control mice. Change in freezing to CS− across the training was significantly higher in CBPΔHAT^{PFC} when compared to control mice. (J) Average learning curves for learning of appropriate responses to CS+ and CS− were calculated based on the performance of control and CBPΔHAT^{PFC} groups across the entire training (Trial Blocks 1-6) (FIG. 1F,G) followed by fitting the regression line and t-test analysis on the mean of those slopes. The analysis of patterns of responses to CS+ and CS− in control animals tested on the context fear discriminatory task revealed that the improvement of fear memory accuracy was due to an incline in freezing to CS+ and a slight decline in freezing to CS− (CS+/Ctrl, α=4.76±1.07; CS−/Ctrl, α=20.88±1.34). The learning of appropriate responses to CS+ shows a positive slope (α) in both control and CBPΔHAT^{PFC} mice and there is no difference between groups (CS+/Ctrl, α=4.76±1.07; CS+/CBPΔHAT^{PFC}, α=6.35±1.61; CS+ slope/Ctrl vs. CBPΔHAT^{PFC} t-test, t₍₁₈₎=20.778, P=0.446). The CBPΔHAT^{PFC} group, which failed to improve fear memory accuracy, showed a positive slope for CS−, a marked difference from control responses to the CS− (CS−/Ctrl, α=20.88±1.34; CS−/CBPΔHAT^{PFC}, α=4.26±1.4); CS− slope/Ctrl vs. CBPΔHAT^{PFC} t-test, t₍₁₈₎=22.614, P=0.018). The asterisks indicate statistical significance: (*) P, 0.05, (**) P, 0.01, (n.s.) not significant.

FIG. 2A-D shows that pavlovian fear conditioning and locomotor activity are normal in CBPΔHAT^{PFC} mice. (A) Pavlovian cued fear conditioning was normal in CBPΔHAT^{PFC} mice. CBPΔHAT^{PFC} and control (Ctrl) mice showed normal acquisition and retention of contextual fear conditioning. Contextual fear was tested in context A at 24 h after the 5 CS-US pairing (CS, 2 s 2800 Hz tone; US-foot shock). CBPΔHAT^{PFC} mice, n=10. Control, n=10. (B-C) Non-induced locomotor activity and (D) anxiety-related responses were unaltered in CBPΔHAT^{PFC} mice.

FIG. 3 shows an experimental design for the auditory discrimination test. The auditory discrimination task tests the ability of subjects to recognize a direction of FM-sweeps ((trains of upward and downward FM-sweeps). The conditioned stimuli (CS) for auditory fear conditioning were 20-s trains of FM-sweeps for a 400-ms duration, logarithmically modulated between 2 and 13 kHz (upsweep) or 13 and 2 kHz (downsweep) delivered at 1 Hz at 75 dB. As described in methods, these assay includes 3 phases: FM-sweep conditioning (day 1-3), generalization (day 4-5) and FM-sweep direction discrimination training (Day 6-12).

FIG. 4A-J shows FM-sweep direction fear memory specificity is deficient in CBPΔHAT^{PFC} mice. (A-B) Pavlovian FM-sweep fear conditioning was normal in CBPΔHAT^{PFC} and mCREB^{PFC} mice. CBPΔHAT^{PFC} and mCREB^{PEC} mice showed similar acquisition (A) and retention (B) of FM-sweep fear conditioning to control (Ctrl) mice. FM-sweep fear was tested in context C at 24 h after a three upsweeps-foot shock pairing. (C) All three groups (CBPΔHAT^{PFC} and mCREB^{PEC} and Ctrl) show no difference in the freezing responses to CS⁺ and CS⁻ (p>0.05) during day 4 and 5 of training, indicating that initially, the CBPΔHAT^{PFC} and mCREB^{PEC} mice generalized responses and did not discriminate between upsweep and downsweep. (D) After the initial generalization of fear conditioned responses, control mice exhibited robust fear memory specificity. (E) CBPΔHAT^{PFC} mice did not discriminate between upsweep and downsweep and exhibited a deficit in auditory fear memory specificity. CBPΔHAT^{PFC} mice demonstrated strong deficit in auditory memory specificity when compared to controls (FIG. 1 b-c, RM-ANOVA, Treatment×context×trial blocks 1-6: F_{(2.806, 81.366)}=3.033, p=0.037). (F) Similarly to CBPΔHAT^{PFC}, mCREB^{PEC} mice did not discriminate between upsweep and downsweep and exhibited a deficit in auditory fear memory specificity. (G) The FM-sweep direction discrimination ratios (DI) were calculated using the freezing responses to CS+ and CS− according to the formula DI=((Upsweep−Downsweep)/(Upsweep+Downsweep)). Analyses revealed differences between CBPΔHAT^{PFC} and control mice in the performance during Days 11-12 between CBPΔHAT^{PFC} and control mice. CBPΔHAT^{PFC}, n=15; Ctrl, n=16. (H) Analyses revealed differences between mCREB^{PEC} and control mice in the performance during Days 11-12. mCREB^{PFC}, n=14; Ctrl, n=16). (I) Change in freezing across training (freezing delta), calculated as the (freezing on Day 12-freezing on Day 7). There was no difference in responses to conditioned stimuli CS+ between CBPΔHAT^{PFC}, mCREB^{PEC} and control mice. Change in freezing to CS− across the training was significantly higher in CBPΔHAT^{PFC} and mCREB^{PFC} when compared to control mice. (J) Average learning curves for learning of appropriate responses to CS+ and CS− were calculated based on the performance of control and CBPΔHAT^{PFC} group across the entire training (FIG. 4D-F; Days 7 to 14) followed by fitting the regression line and t-test analysis on the mean of those slopes (a). The analysis of patterns of responses to CS+ and CS− in control animals tested on the FM-sweep direction fear discriminatory task revealed that the improvement of auditory fear memory accuracy was due to slight incline in freezing to CS+ and rapid decline in freezing to CS− (CS+/Ctrl: α=2.366±0.82; CS−/Ctrl: α=−6.176±1.22). There was no difference in the learning (slopes) of appropriate responses to CS+ between CBPΔHAT^{PFC} and control groups (CS+/Ctrl: α=2.366±0.82; CS+/CBPΔHAT^{PEC}: α=2.384±0.894; CS+ slope/Ctrl vs CBPΔHAT^{PFC} t-test: t₍₂₉₎=−0.015, p=0.988) or mCREB^{PFC} and control mice (CS+/Ctrl: α=2.366±0.82; CS+/mCREB^{PEC}: α=−0.278±1.15; CS+ slope/Ctrl vs mCREB^{PFC} t-test: t₍₂₈₎=1.906, p=0.067). The CBPΔHAT^{PFC} group, which failed to improve fear memory accuracy, showed a positive slope for CS−, a marked difference from control responses to the CS− (CS−/Ctrl: α=−6.176±1.22; CS−/CBPΔHAT^{PFC}: α=−1.22±0.78; CS− slope/Ctrl vs CBPΔHAT^{PFC} t-test; t₍₂₉₎=−3.368, p=0.002). Similar to the CBPΔHAT^{PFC} group, the mCREB^{PFC} group did not improve performance on the auditory discrimination task and showed a positive slope for CS−, a marked difference from control responses to the CS− (CS−/Ctrl: α=−6.176±1.22; CS−/mCREB^{PFC}: α=−0.746±1.03; CS− slope/Ctrl vs mCREB^{PFC} t-test: t₍₂₈₎=−3.347, p=0.002). The asterisks indicate statistical significance: *, p<0.05, **, p<0.01, ***, p<0.001 and n.s. indicates not significant.

### DETAILED DESCRIPTION

As used herein and in the appended claims, the singular forms “a,” “and,” and “the” include plural referents unless the context clearly dictates otherwise. Thus, for example, reference to “an agent” includes a plurality of such agents and reference to “the cognitive function” includes reference to one or more such cognitive functions, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

The publications discussed above and throughout the text are provided solely for their disclosure prior to the filing date of the present application. Nothing herein is to be construed as an admission that the inventors are not entitled to antedate such disclosure by virtue of prior disclosure. Moreover, with respect to any term that is presented in one or more publications that is similar to, or identical with, a term that has been expressly defined in this disclosure, the definition of the term as expressly provided in this disclosure will control in all respects.

CREB (cAMP response element-binding) is a cellular transcription factor. It binds to certain DNA sequences called cAMP response elements (CRE), thereby increasing or decreasing the transcription of the downstream genes. CREB was first described in 1987 as a cAMP-responsive transcription factor regulating the somatostatin gene. Some of the genes whose transcription is regulated by CREB include: c-fos, the neurotrophin BDNF (Brain-derived neurotrophic factor), tyrosine hydroxylase, and many neuropeptides (such as somatostatin, enkephalin, VGF and corticotropin-releasing hormone).

CREB is closely related in structure and function to CREM (cAMP response element modulator) and ATF-1 (activating transcription factor-1) proteins. CREB proteins are expressed in many animals, including humans. CREB has a well-documented role in neuronal plasticity and long-term memory formation in the brain.

The cAMP response element (CRE) is the response element for CREB. Since the effects of protein kinase A on the synthesis of proteins work by activating CREB, the cAMP response element (CRE) is responsible for modulating the effects of protein kinase A that work by protein synthesis.

A typical (albeit somewhat simplified) sequence of events is as follows. A signal arrives at the cell surface, activates the corresponding receptor, which leads to the production of a second messenger such as cAMP or Cat, which in turn activates a protein kinase. This protein kinase translocates to the cell nucleus, where it activates a CREB protein. The activated CREB protein then binds to a CRE region, and is then bound to by a CREB-binding protein (CBP), which coactivates it, allowing it to switch certain genes on or off. The DNA binding of CREB is mediated via its basic leucine zipper domain.

CREB has many functions in many different organs, however most of its functions have been studied in relation to the brain. CREB proteins in neurons are thought to be involved in the formation of long-term memories. CREB is necessary for the late stage of long-term potentiation. CREB also has an important role in the development of drug addiction. There are activator and repressor forms of CREB. Flies genetically engineered to overexpress the inactive form of CREB lose their ability to retain long-term memory. CREB is also important for the survival of neurons, as shown in genetically engineered mice, where CREB and CREM were deleted in the brain. If CREB is lost in the whole developing mouse embryo, the mice die immediately after birth, again highlighting the critical role of CREB in promoting survival.

Disturbance of CREB function in brain can contribute to the development and progression of Huntington's Disease. Abnormalities of a protein that interacts with the KID domain of CREB, the CREB-binding protein (CBP) is associated with Rubinstein-Taybi syndrome. CREB is also thought to be involved in the growth of some types of cancer.

CREB-binding protein, also known as CREBBP or CBP, is a protein that in humans is encoded by the CREBBP gene. The CREB protein carries out its function by activating transcription, where interaction with transcription factors is managed by one or more of p300 domains: the nuclear receptor interaction domain (RID), the CREB and MYB interaction domain (KIX), the cysteine/histidine regions (TAZ1/CH1 and TAZ2/CH3) and the interferon response binding domain (IBiD). The CREB protein domains, KIX, TAZ1 and TAZ2, each bind tightly to a sequence spanning both transactivation domains 9aaTADs of transcription factor p53. Mutations in this gene cause Rubinstein-Taybi syndrome (RTS). Chromosomal translocations involving this gene have been associated with acute myeloid leukemia.

This gene is ubiquitously expressed and is involved in the transcriptional coactivation of many different transcription factors. First isolated as a nuclear protein that binds to cAMP-response element-binding protein (CREB), this gene is now known to play critical roles in embryonic development, growth control, and homeostasis by coupling chromatin remodeling to transcription factor recognition. The protein encoded by this gene has intrinsic histone acetyltransferase activity and also acts as a scaffold to stabilize additional protein interactions with the transcription complex. This protein acetylates both histone and non-histone proteins. This protein shares regions of very high-sequence similarity with protein EP300 in its bromodomain, cysteine-histidine-rich regions, and histone acetyltransferase domain. Recent results suggest that novel CBP-mediated post-translational N-glycosylation activity alters the conformation of CBP-interacting proteins, leading to regulation of gene expression, cell growth and differentiation.

Despite uncertainty in respect to how CBP controls neuronal function via its interaction with multiple regulatory proteins and acetyltransferase activity, the disclosure demonstrates that CBP is an important component of the neural signaling underlying cognitive functioning. However it is difficult to separate developmental defects, compensatory developmental effects and acute function in the adult brain of a gene with pronounced developmental functions. To avoid developmental confounds, four independent manipulations to downregulate CBP acetyltransferase activity specifically in the adult living brain have been reported to date. In addition, ablation of CBP in adult brain resulted in impaired environmental enrichment-induced neurogenesis, which suggest additional role of CBP in adult neurogenesis-dependent enhancement of adaptability toward novel experiences. These data strongly implicate CBP acetyltransferase activity in neural epigenetic signaling underlying long-term memory consolidation.

The disclosure provides methods and compositions for assaying agents for modifying cognitive behavior. In one embodiment, the disclosure provides a non-human animal comprising a reduction or inhibition of a CBP function (“CBPΔHAT^{PFC} organism”). In one embodiment, the gene encoding CBP is disrupted in a sequence corresponding to the histone acetyltransferase domain of CBP.

As used herein a CBPΔHAT^{PFC} non-human animal refers to a non-human animal that has a modified CPB gene in the prefrontal cortex. The CPB is modified through gene therapy and is not inheritable but stably expressed. Non-human organisms having a CBPΔHAT^{PFC} have a reduced acetylation of histones in the PFC.

The methods used for generating genetically modified non-human animals (such as mice) are well known to one of skill in the art and are described in the examples below. The nonhuman animal may be transfected with a suitable vector which contains an appropriate piece of genomic clone designed for homologous recombination.

Numerous methods have been developed over the last decade for the transduction of genes into mammalian cells for potential use in gene therapy. In addition to direct use of plasmid DNA to transfer genes, retroviruses, adenoviruses, parvoviruses, and herpesviruses have been used (Anderson et al., 1995; Mulligan, 1993; the contents of which are incorporated in their entirety into the subject application). Retroviruses have been the vectors of choice. Advantages are that infection of retroviruses is highly efficient and that the provirus generated after infection integrates stably into the host DNA.

Most current gene therapy protocols use murine retroviral vectors to deliver therapeutic genes into target cells; this process, which is called transduction, mimics the early events of retroviral infection. The crucial difference is that, unlike replication competent retroviruses, the vector genome packaged within the viral coat contains no genes for viral proteins and therefore is incapable of replication. For example, a vector would be designed to have 3′ and 5′ long terminal repeat sequences necessary only for the integration of the viral DNA intermediate into the target host cell chromosome and a packaging signal that allows packaging into viral structural proteins supplied by the packaging line in trans (Miller, 1992; Wilson et al., 1990; The contents of which are incorporated in their entirety into the subject application).

For example, in one embodiment, the disclosure provides a method of generating a CBP deficient non-human animal comprising injecting a viral construct containing a CBPΔHAT coding sequence into the medial pre-frontal cortex of the non-human animal. The CBPΔHAT^{PFC} non-human animal is then treated with a test agent followed by challenge with an auditory or context stimuli. The results are compared to (i) the same animal challenged with the auditory or context stimuli prior to treatment with the test agent or (ii) a control set of animals comprising a CBPΔHAT^{PFC} that were not treated with the test agent and which are challenged with the auditory and/or context stimuli. A change in response to the stimuli compared to the control is indicative of an agents that modulates learning and/or fear.

The non-human animal that can be modified to have a modified or non-functional CPB include any non-human mammal (e.g., mouse, rat, pig, monkey etc.). These engineered non-human animals can then be used in various test to determine the effect of a drug or agent on learning, fear and other cognitive functions associated with a defective CPB polypeptide.

Behavioral and cognitive deficits may be determined, for example, by examining the performance of a CBPΔHAT^{PFC} non-human animal in a memory or learning test such as the water maze test.

The disclosure also provides for a screening assay for evaluating whether a compound is effective in improving long-term memory in a subject suffering from impaired long-term memory which comprises: (a) administering the compound to a CBPΔHAT^{PFC} non-human animal, and (b) comparing the long-term memory of the CBPΔHAT^{PFC} non-human animal in step (a) with the long-term memory of a CBPΔHAT^{PFC} non-human animal in the absence of the compound so as to determine whether the compound is effective in rescuing the long-term memory defect thereby improving the long-term memory of the subject.

In embodiments of this screening assay, the CBPΔHAT^{PFC} non-human animal subject can be a rat, a mouse, a sheep, a bovine, a canine, a porcine or a primate. In another embodiment, the compound identified by the screening assay is an organic compound, a peptide, an inorganic compound, a lipid or a small synthetic compound. In a further embodiment, the CBPΔHAT^{PFC} non-human animal utilized in the screening assay is a genetically modified to inhibit the histone acylating function of CPB.

In one embodiment, an assay for screening modulators of cognitive function comprises (a) administering a modulator to a CBPΔHAT^{PFC} animal subject comprising a mutation in CPB (e.g., a mutation in the HAT domain of CPB) and (b) monitoring cognitive function of the animal subject. In one embodiment, the animal subject comprises at least one mutation in the histone acetyltransferase (HAT) domain of the cyclic amp-response element binding protein (CBP) enzyme. In another embodiment, the CBPΔHAT^{PFC} animal subject has at least one substitution mutation in the histone acetyltransferase domain. In a further embodiment, the CBPΔHAT^{PFC} animal subject has a substitute mutation at residues 1540 or 1541 of CBP (SEQ ID NO:2). In another embodiment, the cognitive function is memory consolidation. In yet another embodiment, the cognitive function is memory accuracy. In still another embodiment, the cognitive function is memory generalization. In another embodiment, the cognitive function is fear generalization. In yet another embodiment, the cognitive function is contextual discrimination. In still another embodiment, the cognitive function is auditory discrimination.

As described more fully below, the disclosure provides CBPΔHAT^{PFC} non-human animals (e.g., mice) expressing dominant negative CREB binding protein (CBP) with eliminated acetyltransferase activity. The impact of this dominant negative phenotype was tested and the impact of CBP-dependent mechanisms in the medial prefrontal cortex (mPFC) on fear memory accuracy was measured. Evidence from context and auditory discriminatory tasks indicated that the mPFC circuitry is critical for the acquisition of fear memory accuracy necessary for the recognition of subtle differences between aversive and non-aversive stimuli. These data indicate that CBP-dependent signaling in the mPFC is critical for the suppression of fear responses to non-relevant stimuli, which is a necessary process towards improvement of fear memory accuracy.

The methods and compositions of the disclosure can be used to treat or study impaired long-term memory due to amnesia, Alzheimer's disease, amyotrophic lateral sclerosis, a brain injury, cerebral senility, chronic peripheral neuropathy, a cognitive disability, a degenerative disorder associated with a learning and memory deficit, defective synaptic transmission, Down's Syndrome, dyslexia, electric shock induced amnesia, Guillain-Barre syndrome, head trauma, stroke, cerebral ischemia, Huntington's disease, a learning disability, a memory deficiency, memory loss, a mental illness, mental retardation, memory or cognitive dysfunction, multi-infarct dementia, senile dementia, myasthenia gravis, a neuromuscular disorder, Parkinson's disease, Pick's disease, a reduction in spatial memory retention, senility, Tourrett's syndrome, chronic fatigue syndrome, major depression or electroconvulsive therapy.

The disclosure also provides for a method for improving long-term memory storage and retrieval in a subject suffering from a long-term memory defect which comprises administering to the subject a compound capable of reversing a defect in CPB activity in the subject thereby improving long-term memory storage and retrieval.

The disclosure further provides for a method for improving long-term memory in a subject suffering from a long-term memory defect which comprises administering to the subject a compound identified by the screening assay as effective in improving long-term memory.

The disclosure also provides for a method for improving long-term memory in a subject suffering from a long-term memory defect which comprises administering to the subject a compound that modified CPB activity in the prefrontal cortex thereby improving long-term memory in the subject. In another embodiment, the disclosure provides for a method for improving long-term memory in a subject suffering from a long-term memory defect which comprises administering to the subject an amount of a compound that modifies a CPB CREB biochemical pathway in the frontal cortex of the subject, effective to modify such pathway and thereby improve long-term memory in the subject.

The disclosure encompasses treating a subject suffering from impaired long-term memory. For example, the impaired long-term memory of the subject is due to amnesia, Alzheimer's disease, amyotrophic lateral sclerosis, a brain injury, cerebral senility, chronic peripheral neuropathy, a cognitive disability, a degenerative disorder associated with a learning and memory deficit, defective synaptic transmission, Down's Syndrome, dyslexia, electric shock induced amnesia, Guillain-Barre syndrome, head trauma, stroke, cerebral ischemia, Huntington's disease, a learning disability, a memory deficiency, memory loss, a mental illness, mental retardation, memory or cognitive dysfunction, multi-infarct dementia, senile dementia, myasthenia gravis, a neuromuscular disorder, Parkinson's disease, Pick's disease, a reduction in spatial memory retention, senility, Tourette's syndrome, chronic fatigue syndrome, major depression or electroconvulsive therapy.

In one embodiment, the compound administered to the subject may be an organic compound, a peptide, an inorganic compound, a lipid or a small synthetic compound.

In another embodiment, the subject is a human, a rat, a mouse, a sheep, a bovine, a canine, a porcine or a primate.

In a further embodiment of the disclosure, the administration is via an aerosol, oral delivery, intravenous delivery, an inhalent, an eyedrop, topical delivery, a time-release implant or an intraspinal injection. The implant may be subcutaneous.

The disclosure also provides for a compound identified by the screening assay as effective in improving memory. The compound may be a known compound for which a new use is identified or the compound may be a previously unknown compound.

As used herein, the term “cognitive disorder” includes a learning disability or a neurological disorder which may be Alzheimer's Disease, a degenerative disorder associated with learning, a learning disability, memory or cognitive dysfunction, cerebral senility, multi-infarct dementia and senile dementia, electric shock induced amnesia or amnesia.

The subject may be a mammal or a human subject. The administration may be intralesional, intraperitoneal, intramuscular or intravenous injection; infusion; liposome-mediated delivery; gene bombardment; topical; nasal; oral; anal; ocular or optic delivery.

In the practice of any of the methods or compositions of the disclosure a “therapeutically effective amount” is an amount which is capable of alleviating the symptoms of the cognitive disorder of memory or learning in the subject. Accordingly, the effective amount will vary with the subject being treated, as well as the condition to be treated. For the purposes of the disclosure, the methods of administration are to include, but are not limited to, administration cutaneously, subcutaneously, intravenously, parenterally, orally, topically, or by aerosol.

As used herein, the term “suitable pharmaceutically acceptable carrier” encompasses any of the standard pharmaceutically accepted carriers, such as phosphate buffered saline solution, water, emulsions such as an oil/water emulsion or a triglyceride emulsion, various types of wetting agents, tablets, coated tablets and capsules.

Typically such carriers contain excipients such as starch, milk, sugar, certain types of clay, gelatin, stearic acid, talc, vegetable fats or oils, gums, glycols, or other known excipients. Such carriers may also include flavor and color additives or other ingredients.

This disclosure also provides for pharmaceutical compositions including therapeutically effective amounts of protein compositions and compounds capable of alleviating the symptoms of the cognitive disorder of memory or learning in the subject together with suitable diluents, preservatives, solubilizers, emulsifiers, adjuvants and/or carriers useful in treatment of neuronal degradation due to aging, a learning disability, or a neurological disorder. Such compositions are liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the compound, complexation with metal ions, or incorporation of the compound into or onto particulate preparations of polymeric compounds such as polylactic acid, polglycolic acid, hydrogels, and the like, or onto liposomes, micro emulsions, micelles, unilamellar or multi lamellar vesicles, erythrocyte ghosts, or spheroplasts. Such compositions will influence the physical state, solubility, stability, rate of in vivo release, and rate of in vivo clearance of the compound or composition. The choice of compositions will depend on the physical and chemical properties of the compound capable of alleviating the symptoms of the cognitive disorder of memory or the learning disability in the subject.

Controlled or sustained release compositions include formulation in lipophilic depots (e.g., fatty acids, waxes, oils). Also contemplated are particulate compositions coated with polymers (e.g., poloxamers or poloxamines) and the compound coupled to antibodies directed against tissue-specific receptors, ligands or antigens or coupled to ligands of tissue-specific receptors. Other embodiments of the compositions of the invention incorporate particulate forms protective coatings, protease inhibitors or permeation enhancers for various routes of administration, including parenteral, pulmonary, nasal and oral.

In another embodiments, the disclosure provides methods for screening for a modifier or a modulator of a brain function or a cognitive function. In one embodiment, the disclosure provides methods for modifying or modulating a brain function or a cognitive function in an individual. In another embodiments, methods of the disclosure comprise administering a test compound to a non-human animal having a dysfunctional, non-functional, or partially, substantially or completely disabled CBP or equivalent cellular transcriptional coactivators, and then testing for a change or modulation in a brain function or a cognitive function, such as e.g., an information acquisition capability, short-term or long-term memory, a memory consolidation, a memory accuracy, a memory generalization, a fear generalization, a contextual discrimination, an auditory memory or an auditory discrimination.

The invention will be further described with reference to the examples described herein; however, it is to be understood that the invention is not limited to such examples.

### EXAMPLES

### Example 1

### Exemplary Methods of the Invention

The invention describes methods for screening for compounds or compositions, or genetic modifications, e.g., mutations that can modulate neural mechanisms underlying the attainment of fear memory accuracy for appropriate discriminative responses to aversive and non-aversive stimuli. Considerable evidence indicates that coactivator of transcription and histone acetyltransferase cAMP response element binding protein (CREB) binding protein (CBP) is critically required for normal neural function. CBP hypofunction leads to severe psychopathological symptoms in human and cognitive abnormalities in genetic mutant mice with severity dependent on the neural locus and developmental time of the gene inactivation. The disclosure shows that an acute hypofunction of CBP in the medial prefrontal cortex (mPFC) results in a disruption of fear memory accuracy in mice.

In addition, interruption of CREB function in the mPFC also leads to a deficit in auditory discrimination of fearful stimuli. While mice with deficient CBP/CREB signaling in the mPFC maintain normal responses to aversive stimuli, they exhibit abnormal responses to similar but non-relevant stimuli when compared to control animals. These data indicate that improvement of fear memory accuracy involves mPFC-dependent suppression of fear responses to non-relevant stimuli. Evidence from a context discriminatory task and a newly developed task that depends on the ability to distinguish discrete auditory cues indicated that CBP-dependent neural signaling within the mPFC circuitry is an important component of the mechanism for disambiguating the meaning of fear signals with two opposing values: aversive and non-aversive.

Impairment of Contextual Fear Memory Specificity in CBPΔHAT^{PFC} Mice.

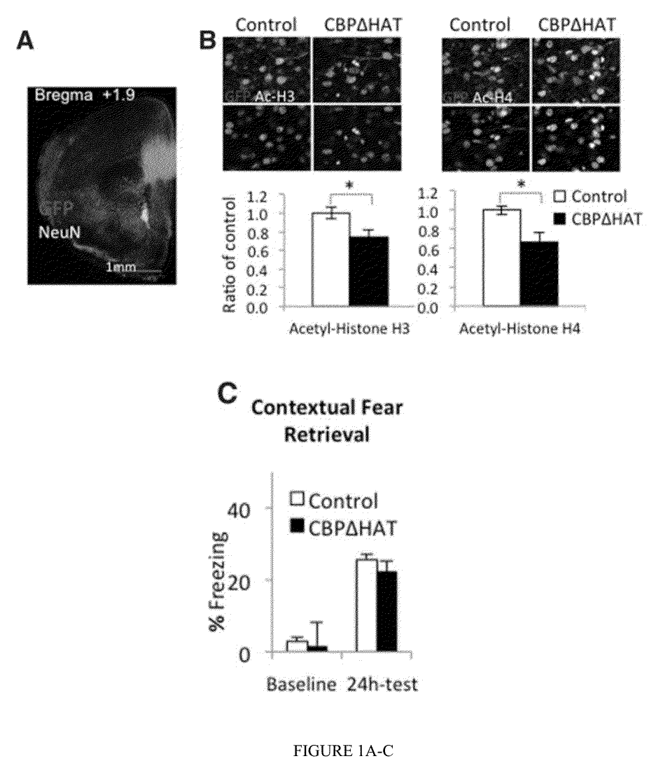

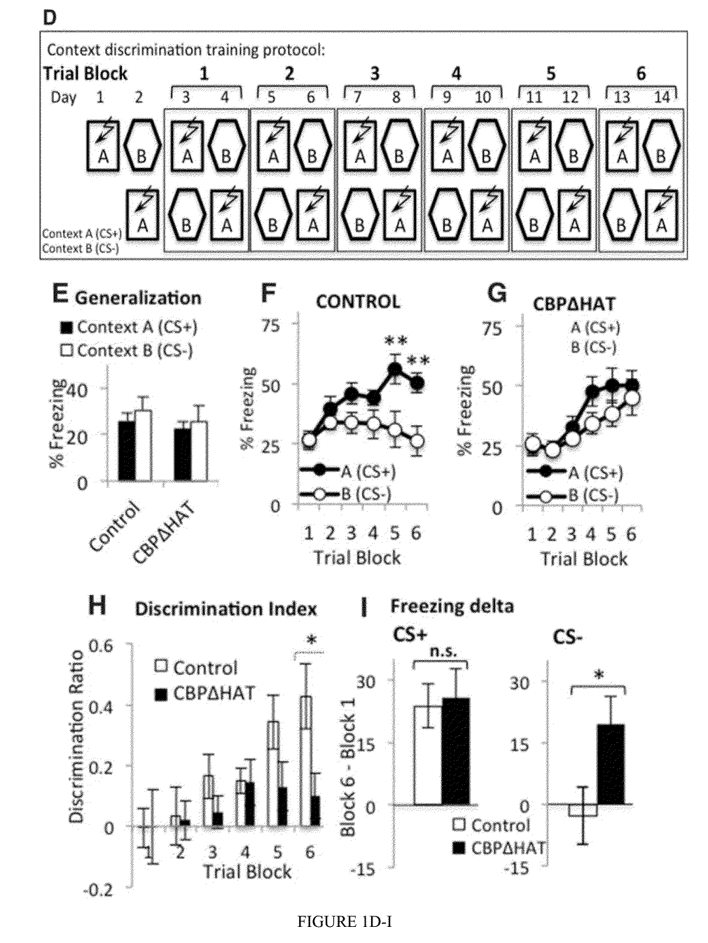

The CBPΔHAT mutant, a dominant-negative inhibitor of CBP-dependent lysine acetylation, harbors a substitution mutation of two conserved residues (Tyr¹⁵⁴⁰/Phe¹⁵⁴¹ to Ala¹⁵⁴⁰/Ala¹⁵⁴¹) in the acetyl CoA binding domain (Korzus et al. 1998; Korzus et al. 2004). This mutant has no intrinsic acetyltransferase activity due to its inability to interact with a donor of acetyl group, acetyl-CoA but retains all protein-protein interaction domains (Korzus et al. 1998). When expressed acutely in adult excitatory neurons, CBPΔHAT functions as a specific blocker of long-term memory consolidation without affecting information acquisition or short-term memory (Korzus et al. 2004). To test the impact of CBP-dependent signaling in the medial prefrontal cortex (mPFC) on fear memory specificity, mice expressing CBPΔHAT and eGFP in the mPFC were generated using virus-mediated gene transfer (referred to as CBPΔHAT^{PFC} mice) (FIG. 1A). For control mice, virus-expressing eGFP only was injected in the mPFC. Cytohistological analysis of brain tissue isolated from CBPΔHAT^{PFC} and control animals revealed that the majority of cells expressing mutant protein in the mPFC were neurons (FIG. 1A-B; Ctrl: 93.85±0.006%, n=3; CBPΔHAT^{PFC}: 92.06±0.012%, n=3; t₍₂₎=−0.03, p=0.511, r=0.013).

CBPΔHAT^{PFC} mice were examined using the fear-conditioning paradigm (FIG. 1C). CBPΔHAT^{PFC} mice performed similar to controls in the contextual version of the fear conditioning task after a 24 h delay (FIG. 1C; Ctrl: 25.78%, n=10; CBPΔHAT^{PFC}: 22.14%, n=10; t₍₁₈₎=1.28, p=0.108). To determine whether the mPFC supports fear memory accuracy, CBPΔHAT^{PFC} mice were also examined using the context fear discrimination task (Lovelace et al. 2014) (FIG. 1D).

First, CBPΔHAT^{PFC} and control mice were tested on a generalization task, in which the freezing responses to novel context B after training on the fear conditioning task to context A was examined. Context B was similar yet not identical to the training context A. No difference in freezing responses to context B or A in CBPΔHAT^{PFC} and control mice was observed (FIG. 1E. Context A vs. B t-test: Ctrl, n=9, p=0.805; CBPΔHAT^{PFC}, n=11, p=0.851). Thus, CBPΔHAT^{PFC} mice did not demonstrate any obvious abnormalities in fear memory generality during the initial presentation of novel context B. Next, CBPΔHAT^{PFC} mice and control littermates were trained to distinguish between the conditioned context A, which was paired with a footshock (CS⁺) and an unconditioned context B, which was not paired with any reinforcement (CS⁻) over multiple training sessions (FIG. 1D). This task requires temporal integration because animals learn subtle differences between context A and B over many days with a single exposure to each context only once per day.

Initially, the control and CBPΔHAT^{PFC} mice generalized their conditioned responses and exhibited similar freezing levels to both the CS⁺ and CS⁻ contexts (block trials 1-4). However, the control animals began to freeze significantly less in response to context B compared to context A after 4 block trials of training, demonstrating the ability to consistently distinguish between similar yet different contexts (block trials 5-6) (FIG. 1F; RM-ANOVA of trial block and context: Context: F_{(1,8)}=9.423, p=0.015; Trial block, F_{(5,40)}=3.24, p=0.015; Trial block×Context: F_{(5,40)}=6.58, p=0.0001; n=9). Post hoc analysis using Bonferroni correction for multiple comparisons indicated that differences were present during trial blocks 5 (p=0.003) and 6 (p=0.005). In contrast to the control animals, CBPΔHAT^{PFC} mice failed to distinguish between context A and B and continued to generalize their conditioned responses throughout all 12 days of training (FIG. 1G, RM-ANOVA of trial block and context: Context: F_{(1,10)}=5.42, p=0.04; Trial block: F_{(2,15)}=11.09, p=0.002; Trial Block×Context: F_{(3,27)}=1.62, p=0.21; n=11).

These data demonstrated that CBPΔHAT expressed in the mPFC resulted in imbalanced neural processes underlying fear memory specificity and generalization. Analysis of the context discrimination ratio confirmed that at the end of the training, the control animals performed better on the context discrimination task compared to the CBPΔHAT^{PFC} mice. FIG. 1H shows no difference in performance between control and CBPΔHAT^{PFC} animals on trial block 1 (t-test: t₍₁₈₎=0.02, p=0.99, r=0.005), but a marked difference on trial block 6 (t-test: t₍₁₈₎=2.60, p=0.018, r=0.52). These findings demonstrate that CBPΔHAT^{PFC} mice have a strong deficit in context discrimination.

Hypothetically, learning of appropriate responses to fearful and similar but not relevant stimuli may involve changes in response to aversive stimuli or non-aversive or both across the entire training. Therefore the fear responses to Context A (CS+) and, separately, to Context B (CS−) in CBPΔHAT^{PFC} and control mice were examined. There was no difference in responses to conditioned stimuli CS+ between CBPΔHAT^{PFC} and control mice across the entire context discrimination training (FIG. 1F-G; RM-ANOVA of trial blocks 1-5 and group: Trial Block×Group: F_{(2.7, 47.9)}=1.782, p=0.169). However, CBPΔHAT^{PFC} and control mice responded differently to non-relevant stimuli CS− across training on the context discriminatory task (FIG. 1F-G; RM-ANOVA of trial blocks 1-5 and group: Trial Block×Group: F_{(2.9, 51.6)}=4.919, p=0.005). Change in freezing to CS− across the training (freezing delta) was significantly higher in CBPΔHAT^{PFC} when compared to control mice (FIG. 1I; t-test: t₍₁₈₎=−2.235, p=0.038).

However, calculations of freezing delta consider only performance on trial blocks 1 and 6. In order to include performance of tested animals on each day across the entire training on the contextual discriminatory task (FIG. 1F-G; Trial Blocks 1-6), average slopes (α) of fitted learning curves were measured (FIG. 1J). The learning of appropriate responses to CS+ shows a positive slope in both control (α=4.76±1.07; where α=slope) and CBPΔHAT^{PFC} (α=6.35±1.61) mice and there is no difference between groups (t-test; t₍₁₈₎=−0.778, p=0.446). The learning of appropriate response to CS-shows a negative slope in the control group (α=−0.88±1.34), which significantly improved fear memory accuracy at the end of training (FIG. 1F). In contrast, the CBPΔHAT^{PFC} group, which failed to improve fear memory accuracy across training (FIG. 1G), showed a positive slope for CS− (α=4.26±1.4), a marked difference from control responses to the CS− (CS−/Ctrl: α=−0.88±1.34; CS−/CBPΔHAT^{PFC}: α=4.26±1.4); CS− slope/Ctrl vs CBPΔHAT^{PFC} t-test; t₍₁₈₎=−2.614, p=0.018).

In summary, analysis of patterns of responses to Context A (CS+) and Context B (CS−) in control animals revealed that the improvement of contextual fear memory accuracy was due to increased freezing behavior to the CS+ and a decrease in freezing to CS−. CBP hypofunction in the mPFC altered the ability to learn discriminatory responses to CS+ versus CS− by disrupting the pattern of the learning curve for CS− only. These data demonstrate that the mPFC supports the improvement of contextual fear memory accuracy by controlling acquisition of appropriate responses to non-relevant stimuli.

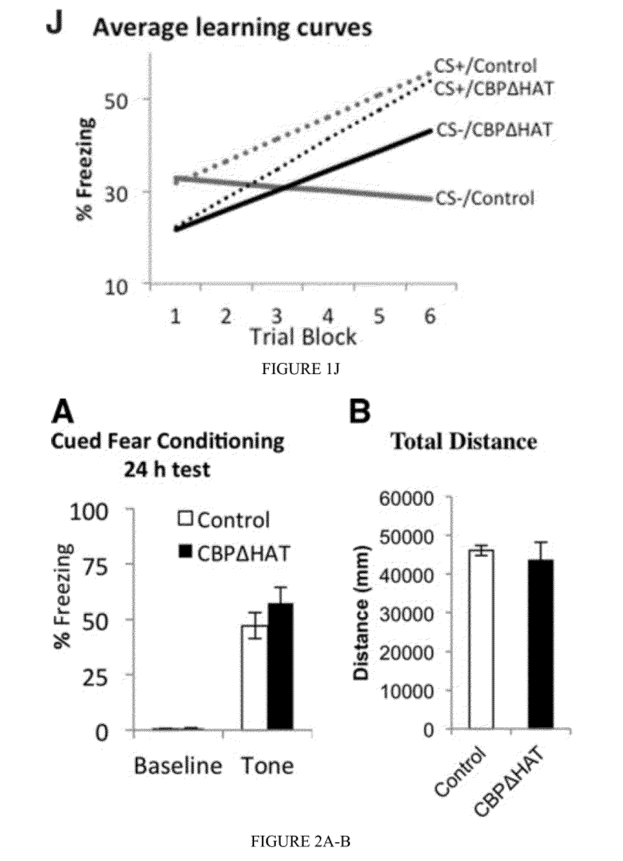

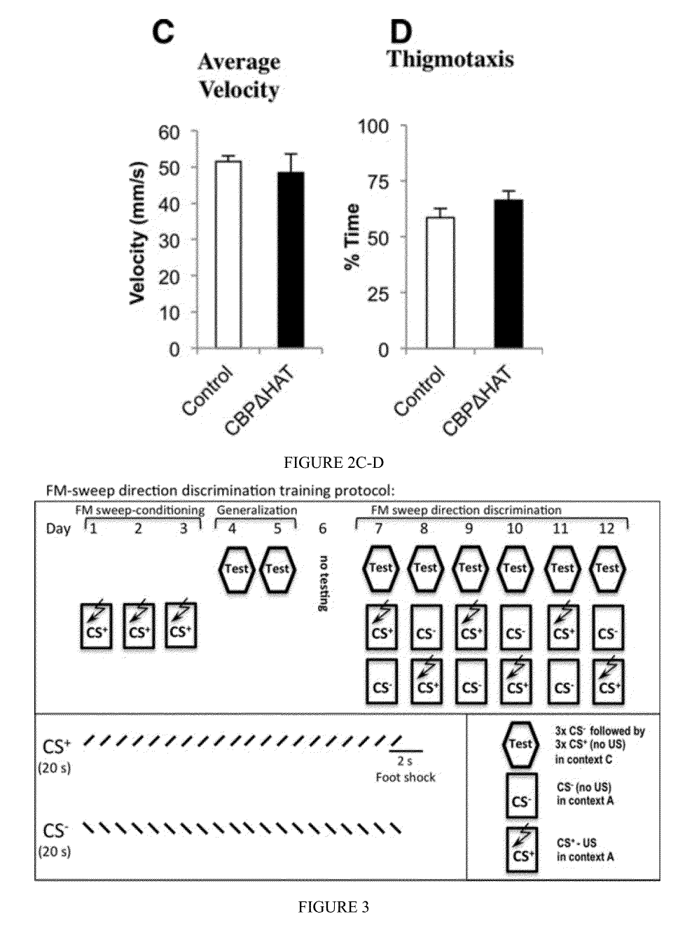

CBPΔHAT^{PFC} mice also performed similar to controls in the cued version of the fear conditioning task during acquisition (data not shown: F_{(5,90)}=1.49, p=0.201) and after a 24 h delay (FIG. 2A. Ctrl: 47.17±5.82%, n=10; CBPΔHAT^{PFC}: 57.27±7.21%, n=10; t₍₁₈₎=−1.042, p=0.324, r=−0.096). These data indicate that information acquisition and long-term memory examined with a 24 hr delay on contextual (FIG. 1C) and cued fear-conditioning (FIG. 2A, 4A-B) were normal in CBPΔHAT^{PFC} mice. The normal performance of CBPΔHAT^{PFC} on these fear-conditioning tasks (FIG. 1C, 2A, 4A-B) indicates that CBPΔHAT^{PFC} mice have functioning circuitry underlying Pavlovian conditioning.

CBPΔHAT^{PFC} mice showed normal levels of locomotor activity (FIG. 2B-D. Total Distance Traveled: Ctrl, 46159.94±1335 mm, n=12; CBPΔHAT^{PFC}, 43563.67±4730.60 mm, n=16; t₍₁₁₎=−0.43, p=0.6627, r=0.1289. Average Velocity: Ctrl: 51.52±1.50 mm/s, n=12; CBPΔHAT^{PFC}: 48.43±5.23 mm/s, n=16; t₍₁₁₎=−0.367, p=0.6399, r=0.1101) and normal anxiety-related responses (FIG. 2E. Thigmotaxis: Ctrl: 58.58±4.12%, n=12; CBPΔHAT^{PFC}: 66.37±6.14%, n=16; t₍₁₁₎=0.34, p=0.3689, r=0.1030).

Impairment of Auditory Memory Specificity in CBPΔHAT^{PFC} Mice.

To evaluate if the deficient discrimination of aversive and non-aversive external stimuli was sensory input-specific, CBPΔHAT^{PFC} mice were examined using a novel auditory discrimination task, which tests the ability of subjects to recognize the direction of frequency modulated (FM)-sweeps (trains of upward and downward FM-sweeps) (FIG. 3). This assay includes 3 days of acquisition (single CS⁺ footshock pairing) followed by a 24 hr test on day 4 and a generalization test on day 4-5. Discrimination training takes place on days 7-12 in which animals are run through 3 sessions: first, they are tested for freezing to CS⁺ and CS⁻ (in context C); second, they are exposed to CS⁺ (or CS⁻); third, they are exposed to CS⁻ (or CS⁺).

In parallel experiments, HSV virus encoding a mutant form of CREB (mCREB) were microinjected into the mPFC and these mice (mCREB^{PEC}) were tested in the auditory discrimination task. CREB is implicated in memory consolidation across variety of species (Dash et al. 1990; Bourtchuladze et al. 1994; Yin et al. 1994; Josselyn et al. 2001; Kida et al. 2002; Pittenger et al. 2002) and functions immediately upstream of CBP. mCREB (CREB^{S133A} mutation) cannot be phosphorylated at the key serine 133 residue and, therefore, cannot recruit CBP and activate transcription (Gonzalez et al. 1989; Chrivia et al. 1993). Thus testing a possible involvement of this well-recognized mediator of memory consolidation in auditory fear discrimination in parallel experiments to those performed in CBPΔHAT^{PFC} mice.

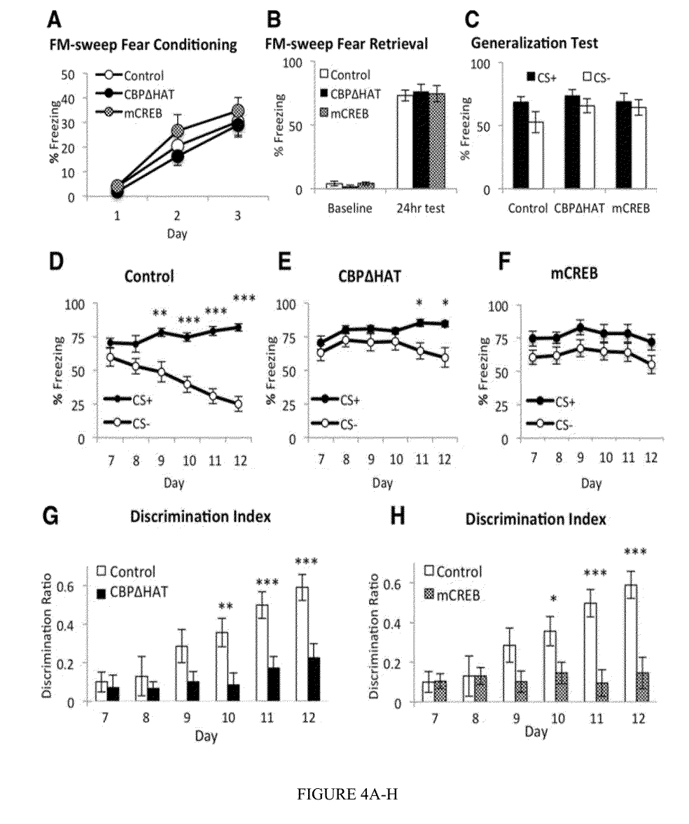

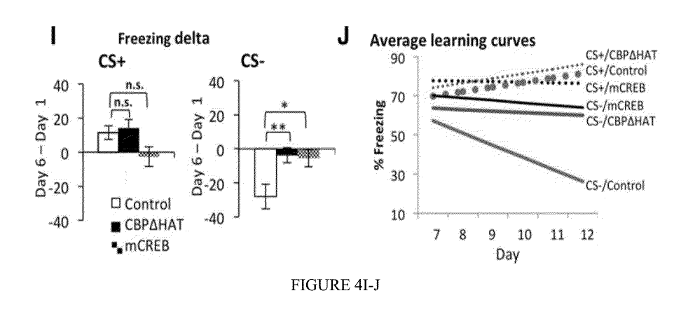

FM-sweep fear conditioning acquisition in CBPΔHAT^{PFC} and mCREB^{PEC} mice was tested. All three groups: the CBPΔHAT^{PFC}, mCREB^{PEC} and control mice similarly acquired this form of Pavlovian conditioning (FIG. 4A; RM-ANOVA of Day and Group: F_{(4,82)}=0.975, p=0.426) and showed the same performance on the 24-hr memory test (FIG. 4B; two way ANOVA of Group and Baseline/24 h-Test; Group: F_{(2,82)}=0.777, p=0.463; Baseline/24 h-Test: F_{(1,82)}=688.3, p=1.2×10⁻⁴¹; Group×Baseline/24 h-Test: F_{(2,82)}=0.205, p=0.815). These data demonstrate that information acquisition and long-term memory tested after a 24-hr delay on FM-sweep fear conditioning was normal in CBPΔHAT^{PFC} and mCREB^{PFC} mice. In addition, CBPΔHAT^{PFC}, mCREB^{PFC} and control mice were also tested on generalization tasks, in which their freezing responses to novel downward FM sweep (CS−) after training on the upward FM-sweep (CS+) fear conditioning task was measured. The generalization test revealed that there was no difference in the freezing responses to the CS− or CS+ between CBPΔHAT^{PFC}, mCREB^{PFC} and control mice (FIG. 4C; ANOVA of FM-sweep direction and group during day 4 and 5: Group: F_{(2,82)}=0.37, p=0.692; ANOVA of FM-sweep direction: F_{(1,82)}=3.458, p=0.067; Group×FM-Sweep Direction: F_{(2,82)}=0.090, p=0.914). These data indicate that strong generalization was observed during days 4 and 5 in all three tested groups.

Next, the animals underwent auditory discrimination training (FIG. 4D-F). Initially, the control, CBPΔHAT^{PFC} and mCREB^{PFC} mice generalized their conditioned responses and exhibited similar levels of freezing responses to both CS⁺ and CS⁻ (days 1-2). However, after 2 days of training, the control animals exhibited a higher number of freezing responses to CS⁺ and significantly fewer freezing responses to CS⁻ compared to CS⁺, demonstrating the ability to consistently distinguish between similar yet different auditory patterns (days 9-12) (FIG. 4D; RM-ANOVA of Day and FM-sweep direction: Day×FM-sweep direction: F_{(2.2,33.5)}=10.776, p=0.0002, n=16). Post hoc analysis using Bonferroni correction (alpha=0.0083) for multiple comparisons indicated that differences were present during days 9 (CS⁺ vs CS⁻ t-test: t₍₃₀₎=3.632, p=0.001, r=0.55), 10 (t₍₃₀₎=5.227, p=0.00001, r=0.69), 11 (t₍₃₀₎=7.540, p=2.1×10⁻⁰⁸, r=0.81) and 12 (t₍₃₀₎=9.253, p=2.7×10⁻¹°, r=0.86) only.

CBPΔHAT^{PFC} mice demonstrated weak ability to discriminate between CS⁺ and CS⁻, and only during the last two days of training (FIG. 4E, RM-ANOVA of Day and FM-sweep direction: Day×FM-sweep direction: F_{(5,70)}=5.071, p=0.001, n=15). Post hoc analysis using Bonferroni correction for multiple comparisons indicated that differences were present during days 11 (CS⁺ vs CS⁻ t-test: t₍₂₈₎=3.149, p=0.004, r=0.51) and 12 (t₍₂₈₎=3.325, p=0.002, r=0.53) only. In contrast to the control animals, CBPΔHAT^{PFC} mice continued to generalize their conditioned responses after 2 days of training and failed to distinguish between context A and B during days 9 and 10 (Day 3: p=0.286; Day 4: p=0.291).

Clearly, CBPΔHAT^{PFC} mice demonstrated strong deficit in auditory memory specificity when compared to controls (FIG. 4D-E, RM-ANOVA of Group and FM-sweep direction and Day 7-12: Group×FM-sweep direction×Day: F_{(2.8,81.4)}=3.033, p=0.037; Group×FM-sweep direction: F_{(1,29)}=7.86, p=0.009; CBPΔHAT^{PFC}, n=15; Ctrl, n=16). Furthermore, analysis of discrimination ratios shows difference in performance between control and CBPΔHAT^{PFC} animals on days 10-12 (FIG. 4G. Discrimination Index CBPΔHAT^{PFC} vs. Ctrl t-test: Day 10: t₍₂₉₎=2.813, p=0.0087, r=0.46; Day 11: t₍₂₉₎=3.546, p=0.001, r=0.55, Day 12: t₍₂₉₎=3.643, p=0.001, r=0.56; CBPΔHAT^{PFC}, n=15; Ctrl, n=16) but not during the initial phase of training. Clearly, control mice show better performance than CBPΔHAT^{PFC} mice on auditory discrimination (FIG. 4D-E, G). Taken together, these data demonstrate that CBPΔHAT expressed in the mPFC resulted in abnormal auditory (FM-sweep direction) fear memory specificity.

Similarly to CBPΔHAT^{PFC} animals, mCREB^{PEC} mice demonstrated a strong deficit in memory specificity during the discrimination phase when compared to controls on the auditory discrimination task (FIG. 3F; RM-ANOVA, Group×FM-sweep direction×Day: F_{(2.8,79.6)}=4.644, p=0.006; mCREB^{PFC}, n=14; Ctrl, n=16). These data demonstrated that mCREB^{PFC} expressed in the mPFC prevented an improvement of auditory memory accuracy across the training as observed in control mice (FIG. 4D). Analysis of the auditory discrimination ratio confirmed that at the end of the training, the control animals performed better on the auditory discrimination task compared to the mCREB^{PFC} mice (FIG. 4H, RM-ANOVA of Day and Group: Day×Group: F_{(2.5,69.0)}=5.149, p=0.005; mCREB^{PFC}, n=14; Ctrl, n=16). Furthermore, analysis of discrimination ratios showed a strong difference in performance between control and mCREB^{PFC} animals on days 10-12 (t-test; day 10: t₍₂₈₎=2.232, p=0.034, r=0.39; day 11: t₍₂₈₎=4.130, p=0.0003, r=0.62; day 12: t₍₂₈₎=4.313, p=0.0002, r=0.63; mCREB^{PFC}, n=14; Ctrl, n=16).

Next, an analysis of fear responses to upsweep (CS+) and, separately, to downsweep (CS−) in control, CBPΔHAT^{PFC} and mCREB^{PFC} mice tested on FM-sweep direction fear discriminatory task were examined (FIG. 4). There was no difference in responses to conditioned stimuli CS+ between CBPΔHAT^{PFC} and control mice across the entire FM-sweep direction discrimination training (FIG. 4D-E; CS+/CBPΔHAT^{PFC} vs Ctrl; RM-ANOVA of days 7-12 and group: Day×Group, F_{(2.8, 81.6)}=0.756, p=0.514). Similarly, there was no difference in responses to conditioned stimuli CS+ between mCREB^{PFC} and control mice across entire FM-sweep direction discrimination training (FIG. 4D, F; CS+/mCREB^{PEC} vs Ctrl; RM-ANOVA of days 7-12 and group: Day×Group: F_{(2.8, 79.5)}=1.808, p=0.155). An analysis of learning curves (FIG. 4J) showed a positive slope to CS+ in control (α=2.366±0.82) and CBPΔHAT^{PFC} (α=2.384±0.894) mice or no change in freezing responses to CS+ in mCREB^{PFC} mice (α=−0.278±1.15) across the entire FM-sweep direction fear discriminatory task. In fact, there was no difference in the learning (slopes) of appropriate responses to CS+ between CBPΔHAT^{PFC} and control groups (FIG. 4J; CS+ slope/Ctrl vs CBPΔHAT^{PFC} t-test; t₍₂₉₎=−0.015, p=0.988) or mCREB^{PFC} and control mice (FIG. 4J; CS+ slope/Ctrl vs mCREB^{PFC} t-test; t₍₂₈₎=1.906, p=0.067). However, CBPΔHAT^{PFC} and mCREB^{PFC} mice responded differently to non-relevant stimuli CS− across training on the auditory discriminatory task when compared to normal mice (FIG. 4D-E; CS−/CBPΔHAT^{PFC} vs Ctrl; RM-ANOVA of days 1-5 and group: Day×Group, F_{(3.8, 111.4)}=6.151, p=0.0002; FIG. 4D,F; CS−/mCREB^{PFC} vs Ctrl; RM-ANOVA of days 1-5 and group: Day×Group: F_{(3.7, 103.8)}=5.685, p=0.0005). When compared to control mice, change in freezing (freezing delta) to CS− across the training was also significantly different in CBPΔHAT^{PFC} (FIG. 4I; t-test: t₍₂₉₎=−2.798, p=0.009) and in mCREB^{PEC} mice (FIG. 4I; t-test: t₍₂₈₎=−2.466, p=0.02). The marked improvement of discrimination observed on the FM-sweep direction fear discriminatory task in control mice (FIG. 4D, G, J) coincides with the significant negative slope of the learning curve for CS− (FIG. 4J; α=−6.176±1.22). The CBPΔHAT^{PFC} group, which failed to improve fear memory accuracy across training (FIG. 4E, G), shows only a slight negative slope for CS− across the training (FIG. 4J; α=−1.22±0.78) and a marked difference when compared to the CS− slope observed in control animals (FIG. 4J; CS− slope/Ctrl vs CBPΔHAT^{PFC} t-test; t₍₂₉₎=−3.368, p=0.002). The mCREB^{PEC} group, which did not improve performance on auditory discrimination task as well (FIG. 4F, H), exhibited similar patterns of learning to the CBPΔHAT^{PFC} mice. While responses to CS+ do not vary from those observed for control mice (FIG. 4I, J), the CS− learning curve is significantly different in mCREB^{PEC} mice compared to control mice (FIG. 4J; CS−/Ctrl: α=−6.176±1.22; CS−/mCREB^{PEC}: α=−0.746±1.03; CS− slope/Ctrl vs mCREB^{PEC} t-test: t₍₂₈₎=−3.347, p=0.002).

In summary, analysis of patterns of responses to CS+ and CS− in control animals tested on the FM-sweep direction fear discriminatory task revealed that the improvement of auditory fear memory accuracy was due to only slight incline in freezing to CS+ and rapid decline in freezing to CS−. CBP hypofunction or CREB hypofunction in the mPFC altered the ability to learn auditory discriminatory responses to CS+ versus CS− by disrupting the pattern of learning for CS− only, while responses to CS+ remained similar to control mice. Consistent with conclusions regarding contextual fear memory specificity, these data demonstrate that the mPFC supports the improvement of auditory fear memory accuracy by controlling acquisition of appropriate responses to non-relevant stimuli.

This invention, and the present findings, provide the first evidence of the critical role that the mPFC plays in the attainment of fear memory accuracy for appropriate discriminative responses to aversive and non-aversive stimuli. This invention, and the present findings, add substantially to the understanding of the circuitry and molecular mechanisms underlying fear memory specificity and generalization.

The data shows that CBP-dependent signaling in the mPFC is required for fear memory accuracy. In addition, fear memory accuracy was also abnormal in mutant mice with disrupted CREB function, which is one of the most widely studied mediators of cellular memory consolidation in Drosophila, Aplysia, and mice (Dash et al. 1990; Bourtchuladze et al. 1994; Yin et al. 1994; Josselyn et al. 2001; Kida et al. 2002; Pittenger et al. 2002). The requirement of CBP acetyltransferase activity for memory consolidation has been demonstrated before including acetylation/deacetylation-targeted pharmacological rescue of memory consolidation in CBPΔHAT mutant mice (Alarcon et al. 2004; Korzus et al. 2004) or late-phase LTP in CBP deficient mutant mice (Alarcon et al. 2004), and also in Aplysia (Guan et al. 2002).

It is important to note that Pavlovian auditory and contextual fear conditioning were intact in CBPΔHAT^{PFC} and mCREB^{PFC} mice. Memory generalization measured immediately after initial fear conditioning was also unchanged in CBPΔHAT^{PFC} and mCREB^{PFC} mice. In addition, there was no difference between tested groups in responses to CS+ across the entire contextual or auditory discriminatory tasks. The abnormal performance of mutant mice in contextual and auditory discriminatory tasks was specific to deficits in responsiveness to CS− only and during later phases of the tasks. These data suggest that prefrontal circuit is critically involved in learning appropriate responses to non-relevant stimuli that are similar yet not identical to aversive stimuli. These data are consistent with the previously described function of the PFC in fear memory extinction. Increasing evidence from human (Kesner and Rogers 2004; Blumenfeld and Ranganath 2007) and animal (Hirsch and Crepel 1992; Morris et al. 1999; Takita et al. 1999; Quirk et al. 2000; Izaki et al. 2002; Maroun and Richter-Levin 2003; Santini et al. 2004; Kawashima et al. 2006; Richter-Levin and Maroun 2010) studies implicate the PFC in extinction of conditioned fear (Sotres-Bayon et al. 2006; Quirk and Mueller 2008) and conditioned taste aversion (Akirav et al. 2006).

There is converging evidence that links fear memory specificity and generality with information processing in the hippocampus-thalamus-PFC-amygdala circuit (Marr 1971; O'Reilly and McClelland 1994; Leutgeb et al. 2007; McHugh et al. 2007; Kumaran and McClelland 2012; Nakashiba et al. 2012; Xu et al. 2012; Navawongse and Eichenbaum 2013; Xu and Sudhof 2013). Involvement of the PFC in context or odor discrimination during information acquisition has been previously studied (Devito et al. 2010; Xu et al. 2012; Xu and Sudhof 2013); however, the contribution of the PFC in the discrimination of auditory patterns, such as FM-sweep direction, has not been previously explored. FM-sweep direction discrimination is important in speech recognition (Zeng et al. 2005) but its underlying neural mechanism is unknown. Auditory fear conditioning has been extensively studied and depends on synaptic plasticity within the amygdala (Fanselow and LeDoux 1999; LeDoux 2000) but neural substrates for auditory fear discrimination is less well studied in mice. Recently, it was suggested that stimulus convergence in the auditory cortex is necessary for the associative fear learning of frequency-modulated sweeps (Letzkus et al. 2011). A reduced reliance on FM-sweep direction stimuli in CBPΔHAT^{PFC} and mCREB^{PFC} mice indicates that the mPFC supports directly auditory fear memory specificity.

There is a general difference in the patterns of freezing responses to CS+/CS− between auditory and context discrimination in control animals. While the direction of learning curves (upwards/downwards) remains the same, their steepness varies. In the context discrimination assay (FIG. 1J), the learning of appropriate responses to CS+ showed a significantly positive slope (FIG. 1J; CS+/Control, α=4.76±1.07; where α=slope), while the learning of appropriate response to CS− showed a slight negative slope (FIG. 1J; CS−/Control, α=−0.88±1.34). The marked improvement of discrimination observed on the FM-sweep direction fear discriminatory task in control mice (FIG. 4D) coincides with the slight positive slope of the learning curve for CS+ (FIG. 4J; CS+/Control, α=2.366±0.82) and the significant negative slope of the learning curve for CS− (FIG. 4J; CS−/Control, α=−6.176±1.22). Two possible factors may have an effect on the steepness of learning curves for acquired responses to CS+/CS− in these discriminatory tasks. First, it is possible that a “floor” effect on CS− curve in the contextual discriminatory task and a “ceiling” effect on CS+ curve in the auditory discriminatory task may account for these differences. Initial level of freezing is substantially lower in the contextual discriminatory task (FIG. 1F-G; ˜25% of initial freezing) when compared to the auditory discrimination task (FIG. 4D-F; above 75% of initial freezing). Second, it may be more difficult to extinguish responses to non-relevant stimuli (Context B) because of high complexity of contextual stimuli (multi-modality, more details). Conversely, the rapid decline of responses to downsweep (CS−) may result from the lower complexity (single modality) of the auditory stimuli and, subsequently, more effective discrimination training.

Recently, it has been proposed that disruption of the PFC circuit during information acquisition may result in over-generality. Inactivation of prefrontal inputs to the nucleus reuniens resulted in an increased fear generalization to novel contextual stimuli (Xu et al. 2012). The manipulation of the mPFC differed and targeted CBP-dependent nuclear processes, which may not produce immediate global effects on firing properties of the mPFC neurons during information acquisition, but rather have effects on the properties of the neural circuits relevant to long-term memory consolidation. However, it is unclear whether the abnormality in fear memory accuracy found in CBPΔHAT^{PFC} mice resulted from fear driven over-generalization or a deficit to access memory details (i.e. memory resolution).

The difficulties with studying CBP function in cognition is confounded by the high complexity of the CBP protein, which can integrate or antagonize multiple signaling pathways and by its distinctive roles in developing and mature circuits. Haploid insufficiency mutations in CBP (Chrivia et al. 1993) or its homolog p300 (Eckner et al. 1994) results in Rubinstein-Taybe syndrome (RTS) (Rubinstein and Taybi 1963; Petrij et al. 1995), which is developmental disorder characterized by severe mental retardation. CBP and p300 both share a very similar molecular structure (Arany et al. 1994) including intrinsic acetyltransferase activity (Ogryzko et al. 1996) and are capable to mediate similar cellular functions including CREB-dependent transcriptional activation. The functional differences between these two redundant genes are due to their highly overlapping but different patterns of expression and not yet understood functional specificity. Prenatal lethality in CBP knockout mice demonstrates an essential role of this gene in embryogenesis (Yao et al. 1998). CBP hemizygote or CBP mutations targeted to excitatory forebrain neurons using CamKIIα promoter driven expression such as conditional knockout or CBPΔHAT^{PFC} mice expressing dominant negative variants display specific deficits in long-term memory but not in short-term memory suggesting that CBP function may support long-term memory encoding. However these results are not consistent across all CBP mutant strains. In one study, CamKIIα-dependent conditional knockout of CBP targeted to excitatory neurons during postnatal brain development resulted in deficient short-term memory (Chen et al. 2010). Although, CamKIIα gene product levels are low during early phases of brain development, a large increase in the expression is usually observed between postnatal days 10 to 30 (Sugiura and Yamauchi 1992; Kojima et al. 1997) coinciding with postnatal brain development. Since the developmental time of CBP conditional deletion was not reported in this study, one cannot eliminate developmental confounds underlying the behavioral phenotype. Thus, it is difficult to dissociate between developmental defects, developmental compensatory effects and acute deficits in mutant mice with CBP hypofunction during critical periods of postnatal brain development. However, when manipulation of CBP activity is performed in the adult brain, data consistently implicate CBP acetyltransferase function in neural epigenetic signaling underlying long-term synaptic plasticity and long-term memory consolidation (Korzus et al. 2004; Barrett et al. 2011; Maddox et al. 2013). In addition, testing of CamKIIα positive cells-restricted and adult mice induced CBP knockout mice indicated that environment-induced adult neurogenesis is extrinsically regulated by CBP function in mature hippocampal granule cells (Lopez-Atalaya et al. 2011). Considering that adult neurogenesis in the hippocampus constitutes an adaptive mechanism to optimally encode contextual information important for memory resolution (Aimone et al. 2011; Sahay et al. 2011) and CBP mutant demonstrates deficiency in spatial discrimination (Lopez-Atalaya et al. 2011) it is likely that CBP is also involved is adult neurogenesis-dependent long term encoding of contextual information. However in CBPΔHAT^{PFC} or mCREB^{PEC} mice hypofunction was targeted to the mPFC and it is unlikely that this manipulation would have an effect on adult neurogenesis in the hippocampus.

How can CBP enzymatic activity regulate neural function? The regulation of gene expression requires not only an activation of transcription factors but also the recruitment of multifunctional coactivators that are independently regulated and directly involved in the chromatin remodeling underlying epigenetic regulatory mechanisms (Rosenfeld and Glass 2001). For example, recent work demonstrated the importance of chromatin remodeling factors like the SWI/SNF complex in neuronal function underlying memory (Vogel-Ciernia et al. 2013). While CBP's function as a platform to recruit other required coactivators appears to be indispensable for CREB-dependent transcription, the recruitment for lysine acetyltransferase activity is transcription unit specific and may depend on the structure of chromatin at a specific locus and/or a specific cell type (Puri et al. 1997; Korzus et al. 1998). Changes in histone acetylation are predictive for gene expression (Allfrey et al. 1964; Pogo et al. 1966). The concordance between the histone acetylation and transcription levels increases over time and the positive correlation between both has been confirmed in genome-wide studies (Kurdistani and Grunstein 2003; Karlic et al. 2010; Markowetz et al. 2010). It is important to emphasize that these are correlations only and that causal relationships between histone modification and gene expression in the brain in vivo will require additional investigation. In addition, a number of non-histone proteins have been identified as substrates for CBP (Kouzarides 2000; Sterner and Berger 2000; Yang 2004; Glozak et al. 2005; Kimura et al. 2005) including CREB (Lu et al. 2003). Regardless of the uncertainty of the CBP's acetyltransferase critical target(s), genetic and pharmacological studies have indicated that hypofunction of CBP's acetyltransferase activity interferes with mechanisms that support memory consolidation and reconsolidation in brain neural networks (Korzus et al. 2004; Maddox et al. 2013). Current data indicate that the acquisition fear memory accuracy involves CBP-dependent mechanism within mPFC circuitry.

Thus, locomotor activity, anxiety-related responses, and fear conditioning were normal in CBPΔHAT^{PFC} mice, yet these mutant mice showed a strong deficit in fear memory accuracy in both contextual and auditory discrimination assays. Both context and auditory fear discrimination tasks required temporal integration because the animals learned subtle differences between relevant and non-relevant stimuli over many days with a single exposure to either CS+ and CS− per day. Inhibition of a component of neural signaling immediately upstream of CBP by a direct blockade of CREB ability to recruit CBP to the target promoter in the mPFC produced identical effects as CBPΔHAT on the capability of mice to learn the distinction between auditory stimuli. Thus, impairment of either component of CREB/CBP-dependent signaling (CREB phosphorylation or CBP's acetyltransferase activity) within the mPFC circuitry resulted in a deficit in auditory fear memory specificity indicating that the mPFC circuitry supports the disambiguation of auditory fear signals.

How CBP and CREB control memory accuracy in the mPFC is unclear. Both CBP and especially CREB have been implicated in long-term plasticity and memory consolidation in Aplysia, Drosophila and mice. Thus it is possible that long term coding within mPFC network involving LTP-mediated modification of prefrontal circuits is critical during contextual and auditory fear discrimination. This type of plasticity in the mPFC might be required to extinguish CS− responses, which would be consistent with the recognized role of the mPFC in fear memory extinction. In addition, CREB has been strongly implicated in adaptive alteration of neuronal excitability and memory allocation (Rogerson et al. 2014) and it is possible that CBP may mediate CREB-dependent changes in neuronal excitability.

There is converging evidence that links contextual fear memory specificity and generality with information processing in the hippocampus-thalamus-PFC-amygdala circuit (Marr 1971; O'Reilly and McClelland 1994; Leutgeb et al. 2007; McHugh et al. 2007; Kumaran and McClelland 2012; Nakashiba et al. 2012; Xu et al. 2012; Navawongse and Eichenbaum 2013; Xu and Sudhof 2013). The findings are consistent with the conclusions reported by DeVito et al., who suggested that the mPFC circuit was critical for the acquisition of overlapping odor discrimination problems (DeVito et al. 2010). Thus, the present findings of the critical role of the mPFC in auditory and context discrimination provides further evidence for the high integration-dependent disambiguation function of the mPFC because similar contexts (or up/down FM-sweeps) were both presented during multiple day training consisting of discontiguous episodes before the animals acquired the ability to properly respond to these signals. These data indicate that certain types of prefrontal dysfunction are likely to contribute to overgeneralized fear, a clinical condition present in anxiety related disorders such as PTSD.

### Materials and Methods

Subjects.

C57BL/6J mice were used for all experiments. Prior to any procedure, the mice are weaned at postnatal day 21, housed 4 animals to a cage with same sex littermates, maintained on a 12 hr light/dark cycle, and had ad libitum access to food and water. Autoclaved bedding was changed every week. All procedures were approved by the UC Riverside Institutional Animal Care and Use Committee in accordance with the NIH guidelines for the care and use of laboratory mice.

Surgery.

The injection protocol has been previously described by Cetin et al. (Cetin et al. 2006). In this study, 2-4-month-old mice were individually housed and weighed to determine the appropriate drug ratios to use. Atropine was injected to help with breathing [0.02 mg/kg body weight]. The mice were then placed into an isoflurane chamber to induce anesthesia, mounted in a heated stereotaxic apparatus and supplied with a constant flow of isoflurane/oxygen mix. The scalp was shaved and sanitized with 70% ethanol. The ear bars, bite bar, and nose clamp were adjusted to firmly hold the head in place. A midline incision was made on the scalp, and surgical hooks were placed to keep the skull exposed. Sterile PBS was added as needed to prevent the skull from drying. The head was leveled by comparing bregma and lambda coordinates until they were equivalent. Injection sites were calculated based on bregma coordinates, and a dental drill was used to thin the skull over the injection site. A 27G needle was then used to remove the thinned bone. A 5-μl calibrated glass micropipette [8 mm taper, 8 μm internal tip diameter] was fitted with a plastic tube connected to a 10-ml syringe and lowered onto a square of Parafilm containing a 4-μl drop of virus. The syringe was aspirated to fill the micropipette with solution before moving it to the injection site. The micropipette was slowly lowered to the proper stereotaxic coordinates and pressure was applied to the syringe to inject 1 μl of solution at a rate of 50 nl/min. After the total volume was injected, the micropipette was withdrawn slowly to avoid backflow, and the injection site was cleaned with sterile cotton swabs. The skin was sutured, and antibiotic was applied to the scalp. Lidocaine was subcutaneously injected near the site followed by an intraperitoneal injection of sterile PBS [30 ml/kg body weight] to prevent dehydration. The mouse was kept warm by placing its cage on a heated plate and injected with buprenorphine [0.05 mg/kg] for pain relief. On post-surgical days 1 and 2, the mouse received subcutaneous injections of meloxicam [1 mg/kg] to relieve pain. Animals were monitored for any signs of distress or inflammation for 3 days after surgery. Behavioral experiments were initiated 3 days after surgery. The infralimbic and prelimbic cortices were targeted at the following stereotaxic coordinates: Bregma; AP 1.8, ML±0.4, DV 1.4.

Viruses.

Surgical procedures were standardized to minimize the variability of HSV virus injections, using the same stereotaxic coordinates for the mPFC and the same amount of HSV injected into the mPFC for all mice. CBPΔHAT or mCREB and/or EGFP were cloned into the HSV amplicon and packaged using a replication-defective helper virus as previously described (Lim and Neve 2001; Neve and Lim 2001). The viruses (HSV/CMV-CBPΔHAT-IRES2-EGFP, HSV/CMV-EGFP and HSV/mCREB-EGFP) were prepared by Dr. Rachael Neve (MIT, Viral Core Facility). The average titer of the recombinant virus stocks was typically 4.0×10⁷ infectious units/ml. HSV viruses are effectively expressed in neurons in the PFC. The CBPΔHAT mutant, a dominant-negative inhibitor of CBP-dependent histone acetylation, harbors a substitution mutation of two conserved residues (Tyr¹⁵⁴⁰/Phe¹⁵⁴¹ to Ala¹⁵⁴⁰/Ala¹⁵⁴¹) in the acetyl CoA binding domain (Korzus et al. 1998). It has been also demonstrated that CBPΔHAT lacks histone acetyltransferase activity (Korzus et al. 2004) and blocks c-fos expression in neurons (Korzus et al. 2004). The dominant negative CREB mutant (mCREB) carries substitution mutation Ser¹³³ to Ala¹³³. Previous studies indicate that mCREB decreased CREB function and block neuronal CREB dependent gene expression (Gonzalez et al. 1989; Chrivia et al. 1993; Barrot et al. 2002; Olson et al. 2005).

Behavioral Assays.

All behavioral experiments were performed under blind conditions.

Fear Conditioning.

Fear conditioning was performed as previously described (Korzus et al. 2004). Fear conditioning training was performed in the fear conditioning box from Coulburn Instruments Inc. After being handled, individual mice were exposed to context A. Context A was the unmodified fear conditioning box, which was placed inside of a sound attenuated chamber with the house light and house fan turned on. Performance was scored by measuring freezing behavior, the complete absence of movement (Fanselow 1980). Freezing was scored and analyzed automatically by a Video-based system (Freeze Frame software ActiMetrics Inc.). Video was recorded at 30 frames per s. The Freeze Frame software calculated a difference between consecutive frames by comparing gray scale value for each pixel in frame. Freezing was defined based on experimenter observations and set as sub-threshold activity for longer than 1 second (s). Freezing was expressed as a % Freezing, which was calculated as a percent of freezing time per total time spent in the testing chamber. The chamber was cleaned in between trials with QUATRICIDE™, 70% ethanol, and distilled water.

Contextual Fear Conditioning.

Mice were trained in a standard Fear Conditioning Chamber Coulburn Instruments Inc.). The individual mice were exposed to context A for 180 s and received a 0.75 mA, 2 s foot shock (context A—foot shock pairing). The animals were then left for another 180 s inside the chamber. For the memory retention test, the mice are placed back into the training chamber for 180 s. Freezing was scored and analyzed automatically as described above.

Cued Fear Conditioning.

Mice were trained in a standard Fear Conditioning Chamber Coulburn Instruments Inc.). After a three-minute baseline period, one, two, or three-20 second tones (2800 Hz, 75 dB) were played and a shock (0.75 mA, 2 sec) was delivered during the final 2 sec of the tone. Twenty-four hours, mice were placed in a novel enclosure and after a three-minute baseline exposure, a series of three tones identical to that given in the training session was played. Freezing was scored and analyzed automatically as described above.

Context Discrimination.

The context discrimination assay was preformed similarly as previously described (Lovelace et al. 2014). After being handled, individual mice were exposed to context A one day before training. The protocol included 14 days of training, which was divided into three phases: initial training phase, generalization test and discrimination phase (FIG. 1D). During the initial training phase (day 1), mice were placed in the context A for 180 s followed by a single foot shock (arrow) and left for another 60 s inside the chamber. Context A (CS+) was the unmodified fear conditioning box (Coulburn Instruments Inc.), which was placed inside of a sound attenuated chamber with the house light and house fan on. The chamber was cleaned with QUATRICIDE™ 70% ethanol, and distilled water. For generalization test and during discrimination phase, the individual mice were exposed to Context A for 180 s and received a 0.75 mA, 2 s foot shock, and left for another 60 s inside the chamber. Four hours later, the mice were exposed to the similar Context B (CS−) for 242 s and received no footshock. Context A and B were similar but not the same. Context B was the modified fear conditioning chamber, with angular wall inserts, house fan off, and scented with Simple Green. Thus animals were exposed to CS⁺ 13 times before the final test. The order of exposure to different contexts was counter balanced. Additionally, the context cues themselves were counter balanced within each group in order to isolate the effect of the CS+.

Auditory Discrimination.

The auditory discrimination task is divided into three phases: initial training phase, generalization test and discrimination phase (FIG. 3). The conditioned stimuli (CS) for auditory fear conditioning were 20-s trains of frequency modulated (FM)-sweeps for a 400-ms duration, logarithmically modulated between 2 and 13 kHz (upsweep) or 13 and 2 kHz (downsweep) delivered at 1 Hz at 75 dB. After habituation, the CS+ was paired with a foot shock (2 s, 0.75 mA). The onset of the US coincided with the onset of the last sweep for the CS. For fear conditioning acquisition (days 1-3; initial training phase), the animals were presented with a single US-CS pairing per day. The FM-sweep Fear Retrieval (day 4) and Generalization (day 4-5) were tested (freezing to 3× CS⁻ for 30 s followed by 3×30 s CS⁺ without US; 3 min baseline and 3 min ITI) in context C, which significantly differed from the training chamber (context A). The discrimination phase of FM sweep direction discrimination training was performed over three sessions a day for 6 days (days 7-12): Session 1 was the performance test, Session 2 was the presentation to 1×UC-CS+ pairing after 3 min baseline, and Session 3 was the presentation to the US-CS− pairing after a 3 min baseline. The CS+ and CS− were counterbalance such that half of the CS+ group was upsweep and the other half CS+ was downsweep.

Open-Field Test.

A 17″×17″×12″ clear Plexiglas arena with a white acrylic floor was used for the open field test. The arena was placed in a sound attenuated chamber with a ceiling mounted camera and a dim light. After sanitizing the arena with QUATRICIDE™, 70% EtOH, and distilled water, the mice were individually placed inside the chamber and allowed to explore for 15 min before being returned to its home cage. Videos are analyzed offline using behavioral analysis software (CleverSys, Inc.) to quantify the level of anxiety and locomotion.

Histology.

Mice were anesthetized using CO₂ and transcardially perfused first with PBS and then 4% PFA. The extracted brain was soaked in 4% PFA overnight and then transferred to PBS until histological sectioning. In this study, 100-μm-thick sections of the mPFC were obtained using a COMPRESSTOME VF-300™ (Precisionary Instr., Greenville, N.C.) and placed in a 24-well plate for free-floating immunohistochemistry (IHC) according to a previously described protocol (Korzus, 2004). The sections are washed 3 times for 10 min in a wash buffer (PBS, 0.3% Triton x-100, 0.02% NaN₂) followed by a 1-hr incubation in blocking buffer (5% normal goat serum in washing buffer), followed by a 10-min incubation in the wash buffer. The sections were incubated overnight at 4° C. with primary antibodies: anti-NeuN (Millipore, Cat No: MAB377). After three washes with the wash buffer, the sections were incubated with secondary antibodies (Alexa647-goat anti-mouse IgG (Molecular Probes, 1:1000), in blocking buffer for 4 hr at room temperature. The sections were washed again three times with the wash buffer before mounting for viewing. Negative control slices were performed for each row of the well plate, undergoing the same IHC procedure in addition to receiving primary antibodies. After immunostaining, the tissue was mounted directly onto glass slides, covered, and sealed with nail polish before imaging.

Imaging.

The slides were placed on the stage of an Olympus FV1000™ laser scanning confocal microscope controlled using the FLUOVIEW™ software. GFP, and Alexa-647 were imaged using 473-nm, and 647-nm lasers, respectively. The background fluorescence was measured and subtracted for each image. The fluorescence intensity was compared to the negative control slices, which did not receive any primary antibodies. Immunostained tissue was analyzed using a semi-automatic laser scanning confocal microscope (Olympus FV1000™) controlled by the FLUOVIEW™ software. Multiple brain sections were imaged using identical microscope settings. Eighty-micrometer z-stacks were obtained from the PL region in the mPFC, and ROI analysis was used for quantification. The background fluorescence was measured for each imaged and then subtracted. The intensity quantification was performed using the FLUOVIEW™ Olympus software and NIH Image J.

Data Analysis.

The experimenters were blind to the group conditions. Data are expressed as the means±SEM. N indicates number of animals unless stated otherwise. Statistical analysis was performed using Excel (Microsoft Inc.) or SPSS (IBM Inc.). The Student's t-test or ANOVA was used for statistical comparisons. Pearson's correlation (r) was used as an effect size. In cases where the repeated measures ANOVA (RM-ANOVA) was utilized and assumptions of sphericity were violated (via Mauchly's Test), the analysis was performed using the Greenhouse-Geisser correction. Where applicable, post hoc analysis with Bonferroni correction was performed for multiple comparisons, which allows for substantially conservative control of the error rate. A p<0.05 was considered statistically significant. The asterisks indicate statistical significance: *, p<0.05, **, p<0.01, ***, p<0.001 and n.s. indicates not significant.

### REFERENCES

Aimone J B, Deng W, Gage F H. 2011. Resolving new memories: a critical look at the dentate gyrus, adult neurogenesis, and pattern separation. Neuron 70: 589-596.
Akirav I, Khatsrinov V, Vouimba R M, Merhav M, Ferreira G, Rosenblum K, Maroun M. 2006. Extinction of conditioned taste aversion depends on functional protein synthesis but not on NMDA receptor activation in the ventromedial prefrontal cortex. Learn Mem 13: 254-258.
Alarcon J M, Malleret G, Touzani K, Vronskaya S, Ishii S, Kandel E R, Barco A. 2004. Chromatin acetylation, memory, and LTP are impaired in CBP+/−mice: a model for the cognitive deficit in Rubinstein-Taybi syndrome and its amelioration. Neuron 42: 947-959.
Allfrey V G, Faulkner R, Mirsky A E. 1964. Acetylation and Methylation of Histones and Their Possible Role in the Regulation of Rna Synthesis. Proc Natl Acad Sci USA 51: 786-794.
Arany Z, Sellers W R, Livingston D M, Eckner R. 1994. E1A-associated p300 and CREB-associated CBP belong to a conserved family of coactivators. Cell 77: 799-800.
Bannister A J, Kouzarides T. 1996. The CBP co-activator is a histone acetyltransferase. Nature 384: 641-643.
Barrett R M, Malvaez M, Kramar E, Matheos D P, Arrizon A, Cabrera S M, Lynch G, Greene R W, Wood M A. 2011. Hippocampal focal knockout of CBP affects specific histone modifications, long-term potentiation, and long-term memory. Neuropsychopharmacology 36: 1545-1556.
Barrot M, Olivier J D, Perrotti L I, DiLeone R J, Berton O, Eisch A J, Impey S, Storm D R, Neve R L, Yin J C et al. 2002. CREB activity in the nucleus accumbens shell controls gating of behavioral responses to emotional stimuli. Proc Natl Acad Sci USA 99: 11435-11440.
Blumenfeld R S, Ranganath C. 2007. Prefrontal cortex and long-term memory encoding: an integrative review of findings from neuropsychology and neuroimaging. Neuroscientist 13: 280-291.
Bourtchuladze R, Frenguelli B, Blendy J, Cioffi D, Schutz G, Silva A J. 1994. Deficient long-term memory in mice with a targeted mutation of the cAMP-responsive element-binding protein. Cell 79: 59-68.
Cetin A, Komai S, Eliava M, Seeburg P H, Osten P. 2006. Stereotaxic gene delivery in the rodent brain. Nat Protoc 1: 3166-3173.
Chen G, Zou X, Watanabe H, van Deursen J M, Shen J. 2010. CREB binding protein is required for both short-term and long-term memory formation. J Neurosci 30: 13066-13077.
Chrivia J C, Kwok R P, Lamb N, Hagiwara M, Montminy M R, Goodman R H. 1993. Phosphorylated CREB binds specifically to the nuclear protein CBP. Nature 365: 855-859.
Dash P K, Hochner B, Kandel ER. 1990. Injection of the cAMP-responsive element into the nucleus of Aplysia sensory neurons blocks long-term facilitation. Nature 345: 718-721.
DeVito L M, Lykken C, Kanter B R, Eichenbaum H. 2010. Prefrontal cortex: role in acquisition of overlapping associations and transitive inference. Learn Mem 17: 161-167.
Eckner R, Ewen M E, Newsome D, Gerdes M, DeCaprio J A, Lawrence J B, Livingston D M. 1994. Molecular cloning and functional analysis of the adenovirus E1A-associated 300-kD protein (p300) reveals a protein with properties of a transcriptional adaptor. Genes Dev 8: 869-884.
Fanselow M S. 1980. Conditioned and unconditional components of post-shock freezing. The Pavlovian journal of biological science 15: 177-182.
Fanselow M S, LeDoux J E. 1999. Why we think plasticity underlying Pavlovian fear conditioning occurs in the basolateral amygdala. Neuron 23: 229-232.
Glozak M A, Sengupta N, Zhang X, Seto E. 2005. Acetylation and deacetylation of non-histone proteins. Gene 363: 15-23.
Gonzalez G A, Yamamoto K K, Fischer W H, Karr D, Menzel P, Biggs W, 3rd, Vale W W, Montminy M R. 1989. A cluster of phosphorylation sites on the cyclic AMP-regulated nuclear factor CREB predicted by its sequence. Nature 337: 749-752.
Gu W, Roeder R G. 1997. Activation of p53 sequence-specific DNA binding by acetylation of the p53 C-terminal domain. Cell 90: 595-606.
Guan Z, Giustetto M, Lomvardas S, Kim J H, Miniaci M C, Schwartz J H, Thanos D, Kandel ER. 2002. Integration of long-term-memory-related synaptic plasticity involves bidirectional regulation of gene expression and chromatin structure. Cell 111: 483-493.
Hirsch J C, Crepel F. 1992. Postsynaptic calcium is necessary for the induction of LTP and LTD of monosynaptic EPSPs in prefrontal neurons: an in vitro study in the rat. Synapse 10: 173-175.
Izaki Y, Takita M, Nomura M. 2002. Local properties of CA1 region in hippocampo-prefrontal synaptic plasticity in rats. Neuroreport 13: 469-472.
Josselyn S A, Shi C, Carlezon W A, Jr., Neve R L, Nestler E J, Davis M. 2001. Long-term memory is facilitated by cAMP response element-binding protein overexpression in the amygdala. J Neurosci 21: 2404-2412.
Karlic R, Chung H R, Lasserre J, Vlahovicek K, Vingron M. 2010. Histone modification levels are predictive for gene expression. Proc Natl Acad Sci USA 107: 2926-2931.
Kawashima H, Izaki Y, Grace A A, Takita M. 2006. Cooperativity between hippocampal-prefrontal short-term plasticity through associative long-term potentiation. Brain Res 1109: 37-44.
Kesner R P, Rogers J. 2004. An analysis of independence and interactions of brain substrates that subserve multiple attributes, memory systems, and underlying processes. Neurobiol Learn Mem 82: 199-215.
Kida S, Josselyn S A, de Ortiz S P, Kogan J H, Chevere I, Masushige S, Silva A J. 2002. CREB required for the stability of new and reactivated fear memories. Nat Neurosci 5: 348-355.
Kimura A, Matsubara K, Horikoshi M. 2005. A decade of histone acetylation: marking eukaryotic chromosomes with specific codes. Journal of biochemistry 138: 647-662.
Kojima N, Wang J, Mansuy I M, Grant S G, Mayford M, Kandel E R. 1997. Rescuing impairment of long-term potentiation in fyn-deficient mice by introducing Fyn transgene. Proc Natl Acad Sci USA 94: 4761-4765.
Korzus E, Rosenfeld M G, Mayford M. 2004. CBP histone acetyltransferase activity is a critical component of memory consolidation. Neuron 42: 961-972.
Korzus E, Torchia J, Rose D W, Xu L, Kurokawa R, McInerney E M, Mullen T M, Glass C K, Rosenfeld MG. 1998. Transcription factor-specific requirements for coactivators and their acetyltransferase functions. Science 279: 703-707.
Kouzarides T. 2000. Acetylation: a regulatory modification to rival phosphorylation? EMBO J 19: 1176-1179.
Kumaran D, McClelland J L. 2012. Generalization through the recurrent interaction of episodic memories: a model of the hippocampal system. Psychological review 119: 573-616.
Kurdistani S K, Grunstein M. 2003. Histone acetylation and deacetylation in yeast. Nat Rev Mol Cell Biol 4: 276-284.
LeDoux J E. 2000. Emotion circuits in the brain. Annu Rev Neurosci 23: 155-184.
Letzkus J J, Wolff S B, Meyer E M, Tovote P, Courtin J, Herry C, Luthi A. 2011. A disinhibitory microcircuit for associative fear learning in the auditory cortex. Nature 480: 331-335.
Leutgeb J K, Leutgeb S, Moser M B, Moser E I. 2007. Pattern separation in the dentate gyrus and CA3 of the hippocampus. Science 315: 961-966.
Lim F, Neve R L. 2001. Generation of high-titer defective HSV-1 vectors. Curr Protoc Neurosci Chapter 4: Unit 4 13.
Lopez-Atalaya J P, Ciccarelli A, Viosca J, Valor L M, Jimenez-Minchan M, Canals S, Giustetto M, Barco A. 2011. CBP is required for environmental enrichment-induced neurogenesis and cognitive enhancement. EMBO J.
Lovelace J W, Vieira P A, Corches A, Mackie K, Korzus E. 2014. Impaired Fear Memory Specificity Associated with Deficient Endocannabinoid-Dependent Long-Term Plasticity. Neuropsychopharmacology.
Lu Q, Hutchins A E, Doyle C M, Lundblad J R, Kwok R P. 2003. Acetylation of cAMP-responsive element-binding protein (CREB) by CREB-binding protein enhances CREB-dependent transcription. J Biol Chem 278: 15727-15734.
Maddox S A, Watts C S, Schafe G E. 2013. p300/CBP histone acetyltransferase activity is required for newly acquired and reactivated fear memories in the lateral amygdala. Learn Mem 20: 109-119.
Mahan A L, Ressler K J. 2012. Fear conditioning, synaptic plasticity and the amygdala: implications for posttraumatic stress disorder. Trends Neurosci 35: 24-35.
Markowetz F, Mulder K W, Airoldi E M, Lemischka I R, Troyanskaya OG. 2010. Mapping dynamic histone acetylation patterns to gene expression in nanog-depleted murine embryonic stem cells. PLoS computational biology 6: e1001034.
Maroun M, Richter-Levin G. 2003. Exposure to acute stress blocks the induction of long-term potentiation of the amygdala-prefrontal cortex pathway in vivo. J Neurosci 23: 4406-4409.
Marr D. 1971. Simple memory: a theory for archicortex. Philosophical transactions of the Royal Society of London Series B, Biological sciences 262: 23-81.
McHugh T J, Jones M W, Quinn J J, Balthasar N, Coppari R, Elmquist J K, Lowell B B, Fanselow M S, Wilson M A, Tonegawa S. 2007. Dentate gyrus NMDA receptors mediate rapid pattern separation in the hippocampal network. Science 317: 94-99.
Morris S H, Knevett S, Lerner E G, Bindman L J. 1999. Group I mGluR agonist DHPG facilitates the induction of LTP in rat prelimbic cortex in vitro. J Neurophysiol 82: 1927-1933.
Nakashiba T, Cushman J D, Pelkey K A, Renaudineau S, Buhl D L, McHugh T J, Rodriguez Barrera V, Chittajallu R, Iwamoto K S, McBain C J et al. 2012. Young dentate granule cells mediate pattern separation, whereas old granule cells facilitate pattern completion. Cell 149: 188-201.
Navawongse R, Eichenbaum H. 2013. Distinct pathways for rule-based retrieval and spatial mapping of memory representations in hippocampal neurons. J Neurosci 33: 1002-1013.
Neve R L, Lim F. 2001. Overview of gene delivery into cells using HSV-1-based vectors. Curr Protoc Neurosci Chapter 4: Unit 4 12.
O'Reilly R C, McClelland J L. 1994. Hippocampal conjunctive encoding, storage, and recall: avoiding a trade-off. Hippocampus 4: 661-682.
Ogryzko V V, Schiltz R L, Russanova V, Howard B H, Nakatani Y. 1996. The transcriptional coactivators p300 and CBP are histone acetyltransferases. Cell 87: 953-959.
Olson V G, Zabetian C P, Bolanos C A, Edwards S, Barrot M, Eisch A J, Hughes T, Self D W, Neve R L, Nestler E J. 2005. Regulation of drug reward by cAMP response element-binding protein: evidence for two functionally distinct subregions of the ventral tegmental area. J Neurosci 25: 5553-5562.
Paxinos G, Franklin K B J. 2001. The mouse brain in stereotaxic coordinates. Academic Press, San Diego.
Peixoto L, Abel T. 2012. The Role of Histone Acetylation in Memory Formation and Cognitive Impairments. Neuropsychopharmacology.
Petrij F, Giles R H, Dauwerse H G, Saris J J, Hennekam R C, Masuno M, Tommerup N, van Ommen G J, Goodman R H, Peters D J et al. 1995. Rubinstein-Taybi syndrome caused by mutations in the transcriptional co-activator CBP. Nature 376: 348-351.
Pittenger C, Huang Y Y, Paletzki R F, Bourtchouladze R, Scanlin H, Vronskaya S, Kandel ER. 2002. Reversible inhibition of CREB/ATF transcription factors in region CA1 of the dorsal hippocampus disrupts hippocampus-dependent spatial memory. Neuron 34: 447-462.
Pogo B G, Allfrey V G, Mirsky A E. 1966. RNA synthesis and histone acetylation during the course of gene activation in lymphocytes. Proc Natl Acad Sci USA 55: 805-812.
Puri P L, Sartorelli V, Yang X J, Hamamori Y, Ogryzko V V, Howard B H, Kedes L, Wang J Y, Graessmann A, Nakatani Y et al. 1997. Differential roles of p300 and PCAF acetyltransferases in muscle differentiation. Mol Cell 1: 35-45.
Quirk G J, Mueller D. 2008. Neural mechanisms of extinction learning and retrieval. Neuropsychopharmacology 33: 56-72.
Quirk G J, Russo G K, Barron J L, Lebron K. 2000. The role of ventromedial prefrontal cortex in the recovery of extinguished fear. J Neurosci 20: 6225-6231.
Richter-Levin G, Maroun M. 2010. Stress and amygdala suppression of metaplasticity in the medial prefrontal cortex. Cereb Cortex 20: 2433-2441.
Rogerson T, Cai D J, Frank A, Sano Y, Shobe J, Lopez-Aranda M F, Silva A J. 2014. Synaptic tagging during memory allocation. Nat Rev Neurosci 15: 157-169.
Rosenfeld M G, Glass C K. 2001. Coregulator codes of transcriptional regulation by nuclear receptors. J Biol Chem 276: 36865-36868.
Rubinstein J H, Taybi H. 1963. Broad thumbs and toes and facial abnormalities. A possible mental retardation syndrome. American journal of diseases of children 105: 588-608.
Sahay A, Wilson D A, Hen R. 2011. Pattern separation: a common function for new neurons in hippocampus and olfactory bulb. Neuron 70: 582-588.
Santini E, Ge H, Ren K, Pena de Ortiz S, Quirk G J. 2004. Consolidation of fear extinction requires protein synthesis in the medial prefrontal cortex. J Neurosci 24: 5704-5710.
Sotres-Bayon F, Cain C K, LeDoux J E. 2006. Brain mechanisms of fear extinction: historical perspectives on the contribution of prefrontal cortex. Biol Psychiatry 60: 329-336.
Sterner D E, Berger SL. 2000. Acetylation of histones and transcription-related factors. Microbiology and molecular biology reviews: MMBR 64: 435-459.
Sugiura H, Yamauchi T. 1992. Developmental changes in the levels of Ca2+/calmodulin-dependent protein kinase II alpha and beta proteins in soluble and particulate fractions of the rat brain. Brain Res 593: 97-104.
Takita M, Izaki Y, Jay T M, Kaneko H, Suzuki SS. 1999. Induction of stable long-term depression in vivo in the hippocampal-prefrontal cortex pathway. Eur J Neurosci 11: 4145-4148.
Valor L M, Pulopulos M M, Jimenez-Minchan M, Olivares R, Lutz B, Barco A. 2011. Ablation of CBP in forebrain principal neurons causes modest memory and transcriptional defects and a dramatic reduction of histone acetylation but does not affect cell viability. J Neurosci 31: 1652-1663.
Valor L M, Viosca J, Lopez-Atalaya J P, Barco A. 2013. Lysine acetyltransferases CBP and p300 as therapeutic targets in cognitive and neurodegenerative disorders. Curr Pharm Des 19: 5051-5064.
Vogel-Ciernia A, Matheos D P, Barrett R M, Kramar E A, Azzawi S, Chen Y, Magnan C N, Zeller M, Sylvain A, Haettig J et al. 2013. The neuron-specific chromatin regulatory subunit BAF53b is necessary for synaptic plasticity and memory. Nat Neurosci.
Wood M A, Kaplan M P, Park A, Blanchard E J, Oliveira A M, Lombardi T L, Abel T. 2005. Transgenic mice expressing a truncated form of CREB-binding protein (CBP) exhibit deficits in hippocampal synaptic plasticity and memory storage. Learn Mem 12: 111-119.
Xu W, Morishita W, Buckmaster P S, Pang Z P, Malenka R C, Sudhof T C. 2012. Distinct neuronal coding schemes in memory revealed by selective erasure of fast synchronous synaptic transmission. Neuron 73: 990-1001.
Xu W, Sudhof T C. 2013. A neural circuit for memory specificity and generalization. Science 339: 1290-1295.
Yang X J. 2004. Lysine acetylation and the bromodomain: a new partnership for signaling. Bioessays 26: 1076-1087.
Yao T P, Oh S P, Fuchs M, Zhou N D, Ch'ng L E, Newsome D, Bronson R T, Li E, Livingston D M, Eckner R. 1998. Gene dosage-dependent embryonic development and proliferation defects in mice lacking the transcriptional integrator p300. Cell 93: 361-372.
Yin J C, Wallach J S, Del Vecchio M, Wilder E L, Zhou H, Quinn W G, Tully T. 1994. Induction of a dominant negative CREB transgene specifically blocks long-term memory in Drosophila. Cell 79: 49-58.
Zeng F G, Nie K, Stickney G S, Kong Y Y, Vongphoe M, Bhargave A, Wei C, Cao K. 2005. Speech recognition with amplitude and frequency modulations. Proc Natl Acad Sci USA 102: 2293-2298.

A number of embodiments of the invention have been described. Nevertheless, it can be understood that various modifications may be made without departing from the spirit and scope of the invention. Accordingly, other embodiments are within the scope of the following claims.

What is claimed is:

1. A method for screening for a modifier or a modulator of a brain function or a cognitive function, comprising:
(a) providing a non-human animal having a dysfunctional, non-functional, or partially, substantially or completely disabled cAMP response element binding protein (CREB) binding protein (CBP) or equivalent cellular transcriptional coactivators,
wherein optionally the brain function or cognitive function comprises information acquisition capability, short-term or long-term memory, a memory consolidation, a memory accuracy, a memory generalization, a fear generalization, a contextual discrimination, an auditory memory or an auditory discrimination,
wherein optionally the non-human animal is a transgenic non-human animal, or a chemically or genetically modified non-human animal, and optionally the non-human animal is a mouse or a rat,
wherein optionally the CBP protein function is partially, substantially or completely disabled by at least one mutation in the CBP gene, and optionally the at least one mutation in the CBP gene comprises at least one mutation in the histone acetyltransferase (HAT) domain of the CBP protein-encoding gene,
and optionally the at least one mutation in the CBP gene comprises at least one mutation in the lysine acetyltransferase (KAT) domain of the CBP protein-encoding gene,
and optionally the mutation results in a CBP that has no intrinsic acetyltransferase activity due to its inability to interact with a donor of acetyl group, acetyl-CoA but retains all protein-protein interaction domains,
and optionally the at least one mutation in the acetyltransferase domain comprises a substitution of residue 1540 or 1541 of SEQ ID NO:2, or equivalent,
wherein optionally the substitution of residue 1540 or 1541 of SEQ ID NO:2, comprises a Tyr¹⁵⁴⁰/Phe¹⁵⁴¹ to Ala¹⁵⁴⁰/Ala¹⁵⁴¹ in the acetyl CoA binding domain;
(b) providing a test compound,
wherein optionally the test compound is a small molecule, a lipid, a nucleic acid, a polysaccharide, peptide or a protein, and optionally the nucleic acid comprises an antisense nucleic acid, or an siRNA or an miRNA; and
(c) administering the test compound to the non-human animal, and testing or determining if the animal has any change in a brain function or a cognitive function,
wherein optionally the brain function or cognitive function comprises information acquisition capability, short-term or long-term memory, a memory consolidation, a memory accuracy, a memory generalization, a fear generalization, a contextual discrimination, an auditory memory or an auditory discrimination,
wherein optionally determining if the animal has any change in a brain function or a cognitive function is accomplished by using a behavioral test or an empirical measurement,
wherein optionally the behavioral test comprises a generalization task or a context fear discrimination task, or a Pavlovian auditory or a contextual fear conditioning,
wherein optionally the empirical measurement comprises use of: a Magnetic resonance imaging (MRI), a nuclear magnetic resonance imaging (NMRI), a magnetic resonance tomography (MRT), a Functional magnetic resonance imaging or functional MRI (fMRI), a Positron emission tomography (PET), a Positron emission tomography-computed tomography (PET-CT or PET/CT), a Electroencephalography (EEG), an Electronystagmography (ENG), or a Magnetoencephalography (MEG), to determine any change in a brain function or a cognitive function,
wherein a finding that the test compound modifies or modulates the brain function or the cognitive function identifies the test compound as a modifier or a modulator of a brain function or a cognitive function.

2. A method for modifying or modulating a brain function or a cognitive function in an individual, comprising:
generating:
(a) a dysfunctional, non-functional, or partially, substantially or completely disabled cyclic AMP-response element binding (CBP) protein or equivalent cellular transcriptional coactivator, or inducing non-expression or dysfunctional expression of, or a dysfunction or non-function in, a CBP protein or equivalent cellular transcriptional coactivator, or
(b) a dysfunctional, non-functional, or partially, substantially or completely disabled nuclear acetyltransferase or histone acetyltransferase, or inducing non-expression or dysfunctional expression of, or a dysfunction or non-function in, a nuclear acetyltransferase or histone acetyltransferase,
by administering a compound or composition or by genetic manipulation of the individual,
wherein optionally compound or composition comprises a small molecule, a lipid, a nucleic acid, a polysaccharide, peptide or a protein, and optionally the nucleic acid comprises an antisense nucleic acid, or an siRNA or an miRNA, and optionally the peptide or a protein comprise an antibody or an antigen binding fragment thereof,
wherein optionally the brain function comprises a cognitive function,
wherein optionally the cognitive function comprises information acquisition capability, short-term or long-term memory, a memory consolidation, a memory accuracy, a memory generalization, a fear generalization, a contextual discrimination, an auditory memory or an auditory discrimination,
wherein optionally the individual is a human or a non-human animal, or the individual is a chemically modified human or a non-human animal, and optionally the non-human animal is a transgenic non-human animal, or a chemically or genetically modified non-human animal, and optionally the non-human animal is a mouse or a rat,
wherein optionally the CBP protein function is partially, substantially or completely disabled by at least one mutation in the CBP gene, and optionally the at least one mutation in the CBP gene comprises at least one mutation in the histone acetyltransferase (HAT) domain of the CBP protein-encoding gene,
and optionally the mutation results in a CBP that has no intrinsic acetyltransferase activity due to its inability to interact with a donor of acetyl group, acetyl-CoA but retains all protein-protein interaction domains,
and optionally the at least one mutation in the histone acetyltransferase (HAT) domain comprises a substitution of residue 1540 or 1541 of SEQ ID NO:2, or equivalent,
wherein optionally the substitution of residue 1540 or 1541 of SEQ ID NO:2, comprises a Tyr¹⁵⁴⁰/Phe¹⁵⁴¹ to Ala¹⁵⁴⁰/Ala¹⁵⁴¹ in the acetyl CoA binding domain.

3. A screening assay for evaluating whether a compound is effective in improving long-term memory in a subject suffering from impaired long-term memory which comprises: (a) administering the compound to the transgenic nonhuman mammal comprising a reduction in histone acetylation by CBP compared to a wild-type non-human mammal, and (b) comparing the long-term memory of the mammal in step (a) with the long-term memory of the mammal in the absence of the compound so as to determine whether the compound is effective in rescuing the long-term memory defect thereby improving the long-term memory of the subject.

4. The screening assay of claim 3, wherein the subject is a human, a rat, a mouse, a sheep, a bovine, a canine, a porcine or a primate.

5. The screening assay of claim 3, wherein the compound is an organic compound, a peptide, an inorganic compound, a lipid or a small synthetic compound.

6. The screening assay of claim 3, wherein the transgenic nonhuman mammal is a genetically modified mouse with reduced or inhibited acetylation of histones in the pre-frontal cortex.

7. The screening assay of claim 3, wherein the impaired long-term memory of the subject is due to amnesia, Alzheimer's disease, amyotrophic lateral sclerosis, a brain injury, cerebral senility, chronic peripheral neuropathy, a cognitive disability, a degenerative disorder associated with a learning and memory deficit, defective synaptic transmission, Down's Syndrome, dyslexia, electric shock induced amnesia, Guillain-Barre syndrome, head trauma, stroke, cerebral ischemia, Huntington's disease, a learning disability, a memory deficiency, memory loss, a mental illness, mental retardation, memory or cognitive dysfunction, multi-infarct dementia, senile dementia, myasthenia gravis, a neuromuscular disorder, Parkinson's disease, Pick's disease, a reduction in spatial memory retention, senility, Tourrett's syndrome, caridac arrest, open heart surgery, chronic fatigue syndrome, major depression or electroconvulsive therapy.

8. A method for improving long-term memory in a subject suffering from a long-term memory defect which comprises administering to the subject a compound identified according to claim 3 that improves long-term memory.

9. The method of claim 8, wherein the impaired long-term memory of the subject is due to amnesia, Alzheimer's disease, amyotrophic lateral sclerosis, a brain injury, cerebral senility, chronic peripheral neuropathy, a cognitive disability, a degenerative disorder associated with a learning and memory deficit, defective synaptic transmission, Down's Syndrome, dyslexia, electric shock induced amnesia, Guillain-Barre syndrome, head trauma, stroke, cerebral ischemia, Huntington's disease, a learning disability, a memory deficiency, memory loss, a mental illness, mental retardation, memory or cognitive dysfunction, multi-infarct dementia, senile dementia, myasthenia gravis, a neuromuscular disorder, Parkinson's disease, Pick's disease, a reduction in spatial memory retention, senility, Tourrett's syndrome, chronic fatigue syndrome, major depression or electroconvulsive therapy.

10. The method of claim 8, wherein the compound is an organic compound, a peptide, an inorganic compound, a lipid or a small synthetic compound.

11. The method of claim 8, wherein the subject is a human, a rat, a mouse, a sheep, a bovine, a canine, a porcine or a primate.

12. The method of claim 8, wherein the administration is via an aerosol, oral delivery, intravenous delivery, an inhalent, an eyedrop, topical delivery, a time-release implant or an intraspinal injection.

13. A compound identified by the screening assay of claim 3 as effective in improving long-term memory.

14. A pharmaceutical composition comprising the compound of claim 13 and a carrier.
